(19) United States
(12) Patent Application Publication
(10) Pub. No.: US 2023/0281244 A1
(43) Pub. Date: Sep. 7, 2023

(54) Audio Content Serving and Creation Based on Modulation Characteristics and Closed Loop Monitoring

(71) Applicant: BrainFM, Inc., Brooklyn, NY (US)

(72) Inventor: Kevin JP Woods, Brooklyn, NY (US)

(21) Appl. No.: 18/177,094

(22) Filed: Mar. 1, 2023

Related U.S. Application Data
(60) Provisional application No. 63/315,485, filed on Mar. 1, 2022.

(51) Int. Cl. G06F 16/635; A61B 5/16; A61M 21/02

(52) U.S. Cl. CPC G06F 16/636; A61B 5/165; A61M 21/02; A61M 2021/0027

(57) ABSTRACT

Disclosed systems and methods include determining a desired trajectory within a multi-dimensional mental state space based on a path from an initial position within the multi-dimensional mental state space to a target position within the multi-dimensional mental state space, where the initial position corresponds to an initial mental state of the user, and where the target position corresponds to a target mental state of the user. Some embodiments include selecting a first media item that has an expected trajectory within the multi-dimensional mental state space that approximates the desired trajectory, and causing playback of the first media item.

### CROSS-REFERENCE TO RELATED APPLICATIONS

This application claims priority to U.S. Provisional App. 63/315,485 titled “Audio Content Serving and Creation Based on Modulation Characteristics and Closed Loop Monitoring,” filed on Mar. 1, 2022, and currently pending. The entire contents of the 63/315,485 application are incorporated by reference. This application is also related to U.S. Pat. No. 7,674,224, titled “Method For Incorporating Brain Wave Entrainment Into Sound Production” and filed Oct. 14, 2005; U.S. Pat. No. 10,653,857, titled “Method To Increase Quality Of Sleep With Acoustic Intervention” and filed Dec. 28, 2017; U.S. Pat. Publication No. 2020/0265827, titled “Noninvasive Neural Stimulation Through Audio” and filed Feb. 15, 2019; U.S. Pat. Application No. 17/366,896, titled “Neural Stimulation Through Audio With Dynamic Modulation Characteristics” and filed Jul. 2, 2021; and U.S. Pat. Application No. 17/505,453, titled “Audio Content Serving And Creation Based On Modulation Characteristics; all of which are incorporated herein by reference.

### BACKGROUND

For decades, neuroscientists have observed wave-like activity in the brain called neural oscillations. Various aspects of these oscillations have been related to mental states including attention, relaxation, and sleep. The ability to effectively induce and modify such mental states by noninvasive brain stimulation is desirable.

### OVERVIEW

Modulation in sound drives neural activity and can support mental states. Sounds that have similar modulation-domain representations may have similar effects on the brain. Analysis of modulation characteristics of pre-existing tracks can allow tracks to be selected to achieve desired mental states, with or without further modification of those tracks. In example embodiments, the present disclosure describes a personalization of audio content based on user-related characteristics and/or the determined effectiveness to a user of similar audio content to achieve a desired mental state.

In example embodiments, the present disclosure provides techniques for serving and creating audio for playback to induce a mental state based on what is effective/ineffective for a user. A measure of effectiveness is not limited to a binary measurement (i.e., effective or ineffective) but can be based on a scale of measurement (e.g. analog rating - X/5 stars, X level of effectiveness as judged by sensors, listening time, etc.).

In example embodiments, the effectiveness of audio in helping the user reach a desired mental state can be determined by user input. Additionally, or alternately, the effectiveness can be determined without an active input by the user. For example, whether an audio was effective to help the user sleep better (desired mental state) can be determined either directly (by asking the user) or indirectly using a smart device such as, for example, an Oura ring (e.g., sleep score, sleep parameters, etc.), an Apple watch (e.g., sleep parameters), a smartphone (e.g., was phone used during ‘sleep time’), etc. In another example, a clinical or academic sleep study performed with participants who are not the user, may be used to determine the effectiveness of an audio track to help the user sleep better. Other examples exist, too.

Another non-limiting example, whether an audio track was effective to help the user stay focused can be determined either directly (by asking the user) or indirectly using a smart device such as a smart watch (e.g., did the user stay seated?), smart phone (e.g., did the user use their phone during focus time?), etc. In yet another non-limiting example, whether an audio track was effective to help the user relax can be determined either directly (e.g., by asking the user) or indirectly using a smart device such as an Oura ring (e.g., was their resting heart rate lower than a threshold?), smart watch (e.g., did their heart rate and blood pressure decrease during a relaxing track?), etc.

In example embodiments, user preference regarding a type of audio can also be taken into consideration. The combination of preferred audio & effective modulation characteristics tailored to a desired mental state may provide a better desired response than an arbitrary audio with modulation characteristics. For example, a user’s preferred music genre (e.g., Country, Jazz, Reggae, Pop, etc.) may be taken into consideration. Alternatively, or additionally, a user’s artist preference (e.g., Willie Nelson, John Coltrane, Bob Marley, Jay-Z, etc.) may be taken into consideration. Alternatively, or additionally, a user’s preferred audio characteristic(s) (e.g., brightness, upbeat, dissonance, etc.) may also be used.

In example embodiments, amplitude modulation analysis can be performed by considering the frequency content of sound envelopes (i.e., the ‘outline’ of broadband or sub band waveforms). Amplitude modulation in sound can drive rhythmic activity in the brain, which may be leveraged to support mental states like focus, sleep, relaxation, meditation, physical exertion (e.g., exercise), and the like. Amplitude modulation analysis is distinct from frequency domain analysis in that the former describes slow rates of change (under 1 kHz) and involves the modulation of a carrier signal whereas the latter describes the sinusoidal components making up the signal itself. Other recommendation systems may not have awareness of modulation-domain analysis (which in the human auditory system involves a modulation-frequency filter bank in the brainstem, similar to the audio-frequency filter bank in the cochlea) and its effects on mental states, and so such recommendation systems may not use modulation-domain analysis and may not target mental states with amplitude modulation.

In example embodiments, modulation-frequency domain analysis (i.e., extraction of modulation characteristics) identifies properties of amplitude fluctuations at rates between 0 Hz - 1000 Hz at any audio frequency, whereas audio-frequency analysis quantifies energy at frequencies across the range of human hearing, from 20 Hz-20 kHz.

In example embodiments, the following techniques can be used for extracting the modulation characteristics from audio: 1) Fast Fourier Transform (fft) of broadband or subband envelopes; 2) modulation domain bandpass filtering; and 3) visual filtering on spectrographic representation. Each of these techniques are described in detail subsequently.

Some example embodiments include: receiving, by a processing device, user-associated data related to a user; determining, by the processing device, one or more desired modulation characteristic values based on the user-associated data; obtaining, by the processing device, a set of one or more target audio tracks, wherein each target audio track represents at least one or more modulation characteristic values; comparing, by the processing device, the desired modulation characteristic values with the modulation characteristic values of at least one target audio track from the set of one or more target audio tracks; selecting, by the processing device, a target audio track from the at least one target audio track based on the comparing, wherein the modulation characteristic values of the target audio track substantially matches the desired modulation characteristic values; and playing, by the processing device, the target audio track.

In various example embodiments, the user-associated data can comprise self-reported user data and/or a target mental state of the user. The self-reported user data can include user information regarding sound sensitivity, age, ADHD and/or preferences for a target audio track and/or preferred audio characteristics. The target mental state can comprise focus, relax, sleep, exercise, and/or meditation. The user-associated data can comprise an audio content with an effectiveness measurement such that the effectiveness measurement indicates an effectiveness of the audio content for the user.

Some example embodiments can include determining, by the processing device, one or more modulation characteristic values of the audio content based on modulation synthesis parameters and/or modulation domain analysis. Other example embodiments can include modifying, by the processing device, the one or more modulation characteristic values of the audio content to match the desired modulation characteristic values based on the effectiveness measurement of the audio content. The modifying can include dynamically modifying modulation characteristics of the audio content.

Some example embodiments can further include selecting, by the processing device, a subsequent target audio track from the set of one or more target audio tracks based on the comparing such that the modulation characteristic values of a beginning portion of the subsequent target audio track aligns in a predetermined manner with an end portion of the target audio track; and chaining, by the processing device, the target audio track and the subsequent target audio track.

Some example embodiments include: receiving, by a processing device, a user’s target mental state; receiving, by the processing device, a reference audio content with an effectiveness measurement that indicates an effectiveness of the reference audio content to achieve the target mental state for the user; determining one or more modulation characteristic values of the reference audio content and/or one or more additional audio parameter values of the reference audio content; obtaining, by the processing device, a set of one or more target audio tracks, wherein each target audio track includes one or more modulation characteristic values and one or more additional audio parameter values; comparing, by the processing device, for at least one target audio track from the set of one or more target audio tracks, the modulation characteristic values of the reference audio content with the modulation characteristic values of the at least one target audio track and the additional audio parameter values of the reference audio content with the additional audio parameter values of the at least one target audio track; and modifying, by the processing device, the at least one target audio track from the set of one or more target audio tracks based on the comparing such that the modulation characteristic values of the at least one target audio track substantially match the modulation characteristic values of the reference audio content and the audio parameter values of the at least one target audio track substantially match the additional audio parameter values of the reference audio content.

In some embodiments, a processing device comprising a processor and associated memory is disclosed. The processing device can be configured to: receive user-associated data related to a user; determine one or more desired modulation characteristic values based on the user-associated data; obtain a set of one or more target audio tracks, wherein each target audio track represents at least one or more modulation characteristic values; compare the desired modulation characteristic values with the modulation characteristic values of at least one target audio track from the set of one or more target audio tracks; select a target audio track from the at least one target audio track based on the comparing, wherein the modulation characteristic values of the target audio track substantially match the desired modulation characteristic values; and play the target audio track.

In some embodiments, a processing device can be configured to: receive a user’s target mental state; receive an audio content with an effectiveness measurement that indicates an effectiveness of the audio content to achieve the target mental state for the user; determine one or more modulation characteristic values of the audio content and one or more additional audio parameter values of the audio content; obtain a set of one or more target audio tracks, wherein each target audio track includes one or more modulation characteristic values and one or more additional audio parameter values; compare, for at least one target audio track from the set of one or more target audio tracks, the modulation characteristic values of the reference audio content with the modulation characteristic values of the at least one target audio track and the additional audio parameter values of the reference audio content with the additional audio parameter values of the at least one target audio track; and modify the at least one target audio track from the set of one or more target audio tracks based on the comparing such that the modulation characteristic values of the at least one audio track substantially match the modulation characteristic values of the reference audio content and the audio parameter values of the at least one target audio track match the additional audio parameter values of the reference audio content.

Some embodiments include determining a desired trajectory within a multi-dimensional mental state space for a user based on a path from an initial position within the multi-dimensional mental state space to a target position within the multi-dimensional mental state space, where the initial position corresponds to an initial mental state of the user, and where the target position corresponds to a target mental state of the user. Some embodiments include selecting a media item (e.g., an audio track, a video track, an audio-video track, or a tactile or other non-auditory or non-visual stimulating track) that has an expected trajectory within the multi-dimensional mental state space that approximates the desired trajectory, and then causing playback of the selected first media item.

In some embodiments, a single media item may include several distinct tracks. For example, a single media item may include (i) several audio tracks that are played serially in a playlist configuration, (ii) an audio track that has an associated video track, where the audio track and video track are played together at the same time, (iii) several video tracks that are played serially in a playlist configuration, (iv) several audio tracks that are played together at the same time, e.g., different channels in a multichannel media item, or (v) any other combination of two or more audio tracks, video tracks, audio-video tracks, or tactile or otherwise non-auditory or non-visual stimulating tracks played serially or in parallel. In operation, a media item containing several distinct media tracks has an expected trajectory that is formed from the combination of expected trajectories of the distinct media tracks comprising the media item.

Some embodiments additionally include, monitoring the user’s mental state while the first media item is playing to determine whether and an extent to which one or both of (i) a current position within the mental state space corresponding to the user’s current mental state is consistent with the desired trajectory and/or (ii) a measured trajectory within the mental state space corresponding to how the user’s mental state has changed over some timeframe is consistent with the desired trajectory.

When the current position and/or measured trajectory is inconsistent with the desired trajectory, some embodiments include determining a revised trajectory to either (i) redirect the user’s current mental state toward the target mental state, or (ii) if the user has previously arrived at the target mental state, return the user to the target mental state.

Based on the revised trajectory, some embodiments include one or both of (i) selecting a second media item that has an expected trajectory within the multi-dimensional mental state space that approximates the revised trajectory, and then transitioning from playback of the first media item to playback of the second media item, or (ii) modifying the expected trajectory of the first media item to approximate the revised trajectory by one or both of (a) modifying one or more audio parameters of the audio track or (b) modifying one or more modulation characteristics of the audio track.

### BRIEF DESCRIPTION OF DRAWINGS

Other objects and advantages of the present disclosure will become apparent to those skilled in the art upon reading the following detailed description of exemplary embodiments and appended claims, in conjunction with the accompanying drawings, in which like reference numerals have been used to designate like elements, and in which:

FIG. 1 is a flowchart of a method according to an example embodiment of the present disclosure;

FIG. 2 shows a waveform of an audio track overlaid with its analyzed modulation depth trajectory according to an example embodiment of the present disclosure;

FIG. 3 is a process flowchart according to an example embodiment of the present disclosure;

FIG. 4A is a process flowchart according to an example embodiment of the present disclosure;

FIG. 4B is a process flowchart according to an example embodiment of the present disclosure;

FIG. 5 is a functional block diagram of a processing device according to an example embodiment of the present disclosure;

FIG. 6 is an example system with various components according to an example embodiment of the present disclosure;

FIG. 7 is an example interface of a closed loop system, according to an example embodiment of the present disclosure;

FIGS. 8A-8B are additional example interfaces of the closed loop system, according to an example embodiment of the preset disclosure; and

FIGS. 9A-9C are additional example interfaces of the closed loop system, according to an example embodiment of the present disclosure.

FIGS. 10A-E show process flowcharts according to example embodiments of the present disclosure.

The figures are for purposes of illustrating example embodiments, but it is understood that the inventions are not limited to the arrangements and instrumentality shown in the drawings. In the figures, identical reference numbers identify at least generally similar elements.

### DESCRIPTION

The present disclosure describes systems, methods, apparatuses and computer executable media for personalizing, for a user, a selection of one or more target audio tracks for playback. The personalizing can be based on one or more of the following aspects: a user-associated data (e.g., target mental state of a user, self-report data, behavioral data for a user, effectiveness ratings of audio tracks previously played by a user, sensor-input values for a sensor associated with a user, etc.), a reference audio track, and modulation characteristics of the one or more target audio tracks, whereby the modulation characteristics can be based on modulation synthesis parameters and/or modulation domain analysis of the one or more target audio tracks. The target audio tracks can be selected for a user based on their effectiveness towards a user’s desired mental state based on their modulation characteristics, rather than mere aesthetic rating and/or music parameters (e.g., tonality, instrumentation, chords, timbre, etc.) as provided by known services.

In example embodiments, modulation characteristics may include depth of modulation at a certain rate, the rate itself, modulation depth across all rates (i.e., the modulation spectrum), phase at a rate, among others. These modulation characteristics may be from the broadband signal or in subbands (e.g., frequency regions, such as bass vs. treble). The subbands used may be based on cochlear subbands (i.e., the frequency decomposition employed at the human auditory periphery). Audio/audio track/audio content, as used herein, can refer to a single audio element (e.g., a single digital file), an audio feed (either analog or digital) from a received signal, or a live recording. As used herein, audio/audio track/audio content can be a temporal portion of an audio track/content (e.g., one or more snippets of an audio track/content), a spectral portion of an audio track/content (e.g., one or more frequency bands or instruments extracted from an audio track/content) or a complete audio track/content.

In the past, technologies have been targeted to the full range of audio frequencies perceptible to humans. The present disclosure describes technologies that may target specific subregions of that range. In various exemplary embodiments described herein, the modulation can be effective when applied at predetermined frequencies, which are associated with known portions of the cochlea of the human ear and may be referenced in terms of the cochlea, or in terms of absolute frequency. For example, predetermined frequencies can be associated with portions of the cochlea of the human ear that are more sensitive for neuromodulation. Additionally, predetermined frequencies can be associated with portions of the cochlea of the human ear that are perceived less sensitively such that the modulation is not distracting to a user. Note that these are specific regions within the full range of human hearing. Furthermore, the presently disclosed techniques may provide for a selection of modulation characteristics configured to target different patterns of brain activity. These aspects are subsequently described in detail.

In various exemplary embodiments described herein, audio can be modulated in order to affect patterns of neural activity in the brain to affect perception, cognition, action, and/or emotion. Modulation can be added to audio (e.g., mixed) which can in turn be stored and retrieved for playback at a later time. Modulation can be added to audio (e.g., mixed) for immediate (e.g., real-time) playback. Modulated audio playback may be facilitated from a playback device (e.g., smart speaker, headphone, portable device, computer, etc.) and may be single or multi-channel audio. Modulated audio playback may be facilitated through a playback device that transforms the audio into another sensory modality such as vibration or modulated light, rather than being an audible signal. Users may facilitate the playback of the modulated audio through, for example, an interface on a processing device (e.g., smartphone, computer, etc.). These aspects are subsequently described in detail.

### A. Selecting Media Items for Playback Based on Desired Modulation Characteristics

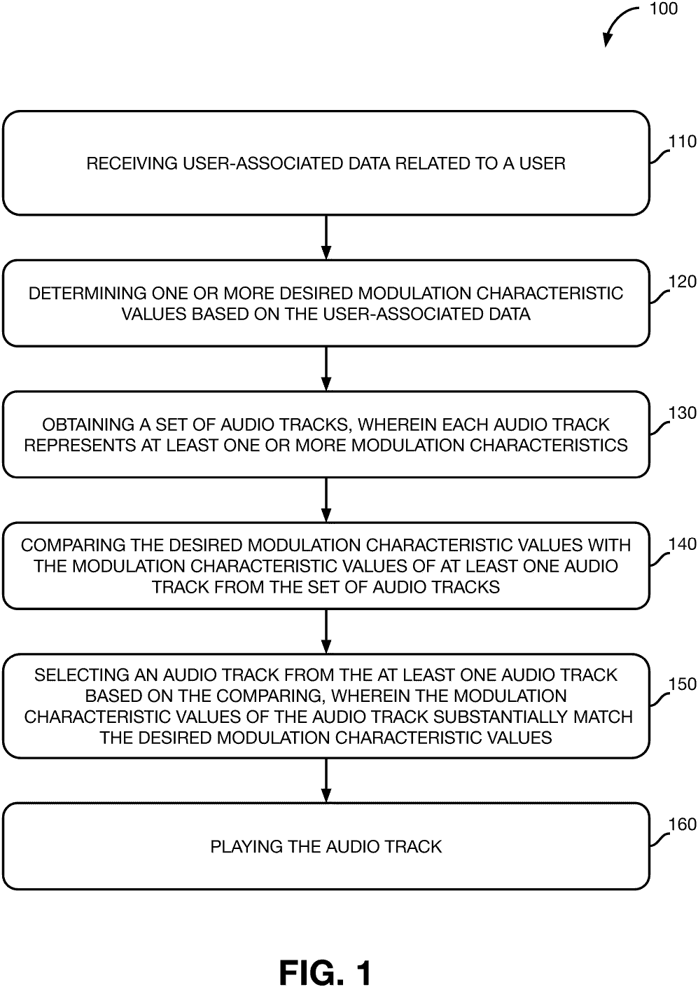

FIG. 1 illustrates an example method 100 performed by a processing device (e.g., smartphone, computer, smart speaker, etc.) according to an example embodiment of the present disclosure. The method 100 may include one or more operations, functions, or actions as illustrated in one or more of blocks 110-160. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation.

Method 100 can include a block 110 of receiving user-associated data. In example embodiments, user-associated data can comprise self-report data such as, for example, a direct report or a survey, e.g., ADHD self-report (e.g., ASRS survey or similar), autism self-report (e.g., AQ or ASSQ surveys or similar), sensitivity to sound (e.g., direct questions), genre preference (e.g., proxy for sensitivity tolerance), work habits re. music/ noise (e.g., proxy for sensitivity tolerance), and/or history with a neuromodulation. Self-report data can also include time-varying reports such as selecting one’s level of relaxation once per minute, leading to dynamic modulation characteristics over time in response. User-associated data can also comprise other user surveys such as, for example, onboarding questions (e.g., questions of users new to the presently disclosed system/methods etc.), personality questionnaires (e.g. questions related to personality of a user), etc.

In example embodiments, user-associated data can comprise effectiveness ratings of audio tracks previously played by a user. The effectiveness ratings can be based on explicit ratings provided by the user (e.g., the user provides a 5-star rating to a track, etc.) or implicit ratings (e.g., a user skipping the track repeatedly reflects a lower rating, a user repeatedly playing a track or submitting a track to the server reflects a higher rating, etc.).

In example embodiments, user-associated data can comprise behavioral data/attributes such as user interests, a user’s mental state, emotional state, etc. User-associated data can include data about the user’s current temporary condition (i.e., states) and/or the user’s unchanging persistent conditions (i.e., traits). User-associated data can be obtained from various sources such as user input, the user’s social media profile, etc. User-associated data can comprise factors external to, but related to, the user such as, for example, the weather at the user’s location; the time after sunrise or before sunset at the user’s location; the user’s location; or whether the user is in a building, outdoors, or a stadium.

In example embodiments, user-associated data can comprise sensor-input values obtained from one or more sensors associated with the user. The sensors may include, for example, an inertial sensor such as an accelerometer (e.g., phone on table registers typing which may be used as a proxy for productivity); a galvanic skin response (e.g., skin conductance); a video or image camera (e.g., user-facing: eye tracking, state sensing; outward-facing: environment identification, movement tracking); and a microphone (e.g., user-sensing: track typing as proxy for productivity, other self-produced movement; outward-sensing: environmental noise, masking, etc.). The sensors may include a physiological sensor such as, for example, a heart rate monitor; a blood pressure monitor; a body temperature monitor; an EEG; a MEG (or alternative magnetic-field-based sensing); a near infrared sensor (fnirs); and/or bodily fluid monitors (e.g., blood or saliva for glucose, cortisol, etc.).

The sensors may include real-time computation. Non-limiting examples of a real-time sensor computation include: the accelerometer in a phone placed near a keyboard on a table registering typing movements as a proxy for productivity; an accelerometer detecting movements and reporting a user has started a run (e.g. by using the CMMotionActivity object of Apple’s iOS Core ML framework), and microphone detecting background noise in a particular frequency band (e.g., HVAC noise concentrated in bass frequencies) and reporting higher levels of distracting background noise. In an example embodiment, where the audio content includes background noise, determining modulation characteristics (described subsequently) can be optional.

The sensors can be on the processing device and/or on an external device and data from the sensor can be transferred from the external device to the processing device. In one example, the sensor on a processing device, such as, for example, an accelerometer on a mobile phone, can be used to determine how often the phone is moved and can be a proxy for productivity. In another example, the sensor on an activity tracker (e.g., external device) such as, for example, an Oura ring or Apple watch, can be used to detect if the user is awake or not, how much they are moving, etc.

In some embodiments, the sensors can be occasional-use sensors used to calibrate the music to stable traits of the user or their environment. For example, a user’s brain response to modulation depth can be measured via EEG during an onboarding procedure which may be done per use or at intervals such as once per week or month. In other embodiments, the sensors can be responsive to the user’s environment. For example, characterizing the acoustic qualities of the playback transducer (e.g., for headphones/speakers) or the room using a microphone, electrical measurement, an audiogram, or readout of a device ID. The sensors can measure environmental factors that may be perceived by the user such as, for example, color, light level, sound, smell, taste, and/or tactile.

In some embodiments, behavioral/performance testing can be used to calibrate the sensors and/or to compute sensor-input values. For example, a short experiment for each individual to determine which modulation depth is best via their performance on a task. Similarly, external information can be used to calibrate the sensors and/or to compute sensor-input values. For example, weather, time of day, elevation of the sun at user location, the user’s daily cycle/circadian rhythm, and/or location. Calibration tests, such as calibrating depth of modulation in the music to individual users’ sound sensitivity based on a test with tones fluctuating in loudness can also be used to calibrate the sensors and/or to compute sensor-input values. Each of these techniques can be used in combination or separately. A person of ordinary skill in the art would appreciate that these techniques are merely non-limiting examples, and other similar techniques can also be used for calibration of the music based on sensors.

In some embodiments, the sensor-input value can be sampled at predetermined time intervals, or upon events, such as the beginning of each track or the beginning of a user session or dynamically on short timescales/real-time (e.g., monitoring physical activity, interaction with phone/computer, interaction with app, etc.).

In example embodiments, user associated data can include one or more of a target mental state for the user (e.g., sleep, focus, meditation, etc.), user-associated inputs (e.g., history of subjective reports, effectiveness ratings of previous tracks, onboarding questions, personality questionnaires, behavioral input, sensor input values, etc.), and modulation characteristics of one or more reference audio tracks.

At block 120, one or more desired modulation characteristics values can be determined based on the user-associated data. In example embodiments, modulation rate, phase, depth, and waveform can be four non-exclusive modulation characteristics. Modulation rate can be the speed of the cyclic change in energy, and can be defined, for example, in hertz. Modulation phase is the particular point in the full cycle of modulation, and can be measured, for example, as an angle in degrees or radians. Modulation depth can indicate the degree of amplitude fluctuation in the audio signal. In amplitude modulation, depth can be expressed as a linear percent reduction in signal power or waveform envelope from peak-to-trough, or as the amount of energy at a given modulation rate. Modulation waveform may express the shape of the modulation cycle, such as a sine wave, a triangle wave, or some other custom wave. These modulation characteristics can be extracted from the broadband signal or from subbands after filtering in the audio-frequency domain (e.g., bass vs. treble), by taking measures of the signal power over time, or by calculating a waveform envelope (e.g., the Hilbert envelope).

In example embodiments, modulation characteristic values in the audio can be determined using various techniques. Non limiting examples of such techniques can include Fast Fourier Transform (FFT) on the envelope (e.g., ‘waveform outline’) techniques, modulation domain bandpass filtering that provides phase and amplitude of modulation, visual filtering on spectrographic representation (e.g., use a spectrogram/cochleagram to run a 2D fourier transform, visual filter like convolution with a gabor patch, etc.), or other known techniques. The FFT and the bandpass filtering techniques can be based on subband envelopes. The visual filtering technique can get subbands via a spectrographic representation.

An example visual filtering of a spectrogram technique is described in: Singh NC, Theunissen FE. Modulation spectra of natural sounds and ethological theories of auditory processing. J Acoust Soc Am. 2003 Dec;114(6 Pt 1):3394-411. doi: 10.1121/1.1624067. PMID: 14714819. An example technique for FFT of subband envelopes is described in: Greenberg, S., & Kingsbury, B. E. (1997, April). The modulation spectrogram: In pursuit of an invariant representation of speech. In 1997 IEEE International Conference on Acoustics, Speech, and Signal Processing (Vol. 3, pp. 1647-1650). IEEE. An example technique for modulation filterbank method described in: Moritz, N., Anemüller, J., & Kollmeier, B. (2015). An auditory inspired amplitude modulation filter bank for robust feature extraction in automatic speech recognition. IEEE/ACM Transactions on Audio, Speech, and Language Processing, 23(11), 1926-1937. All of these publications are incorporated in their entirety by reference.

In example embodiments, to determine the desired modulation characteristics, a user may be asked after playing an audio track, “Did this track help you to focus?” and presented with a (e.g., thumbs-up and thumbs-down) selection to choose a response. The user can then be presented target audio content (as subsequently described with respect to blocks 140-160) that has similar modulation characteristics (i.e., to drive brain rhythms similarly) to tracks they rated (e.g., thumbs-up). Similarly, a user may be asked for access to their personal focus-music playlist (e.g., to be used as reference tracks), which can be analyzed to determine what modulation characteristics the user finds effective.

In example embodiments, to determine the desired modulation characteristics, a smart device may communicate with the processing device to provide an evaluation of the effectiveness of a reference track. In one example, one or more messages are transmitted between a first application running on the processing device to a second application (e.g., Oura, Apple Health, FitBit, etc.) on an external device (e.g., smart device such as a smart ring, watch, phone, etc.). The one or more messages may include, among other possibilities, a specific type of mental state and/or activity (e.g., sleep, focus, run, etc.) and a time interval (e.g., start/end time, absolute time, etc.) to make the evaluation. The external device may in turn send one or more messages to the processing device indicating a determined mental state and/or evaluation (e.g., based on information gathered during the time interval). In some embodiments, the first and second applications may be the same. In some embodiments, the external device can be the same as the processing device.

In example embodiments, a user model can be generated based on user-associated data that can include user-related input and the user’s target mental state. For example, user-related input can be in the form of one or more of (1) information about the user (ADHD, age, listening preferences, etc.); (2) sensor data; and (3) reference tracks with explicit (e.g., stars) or implicit (e.g., provided to the system) rating. A user’s mental state can be explicitly provided, inferred, or assumed by the system.

The user model can be defined over a set of modulation characteristics for a user’s desired mental state. The user model can prescribe regions in the modulation-characteristic space that are most effective for a desired mental state. The user model may be a function defining predicted efficacy of music, in a high-dimensional space, with dimensions of modulation rate, modulation depth, audio brightness and audio complexity. The user model may be based on prior research that relates modulation characteristics to mental states. For example, if the user says they have ADHD and are of a particular age and gender, then the user model may incorporate this information to determine desired modulation characteristics for a particular target mental state of the user. The determination may, for example, be based on a stored table or function which is based on prior research about ADHD (e.g., users with ADHD require a relatively high modulation depth). Another non-limiting example for defining and/or modifying a user model can be based on reference tracks and ratings provided by a user. The reference tracks can be analyzed to determine their modulation characteristics. The determined modulation characteristics along with the ratings of those tracks can be used to define or modify the user model.

In example embodiments, the user model can be updated over time to reflect learning about the user. The user model can also incorporate an analysis of various audio tracks that have been rated (e.g., {for effectiveness {focus, energy, persistence, accuracy}, or satisfaction}, positively or negatively). The inputs to generate a user model can include ratings (e.g., scalar (X stars), binary (thumbs up/down)), audio characteristics (e.g., modulation characteristics, brightness, etc.) For example, a user known to have ADHD may initially have a user model indicating that the target audio should have higher modulation depth than that of an average target track. If a user subsequently provides a reference track with a positive indication, and it is determined that the reference track has a low modulation depth (e.g., 0.2 out of 1), then the target modulation depth may be updated in the user model (e.g., to an estimate that a low depth is optimal). If the user subsequently provides three more reference tracks with positive indications, and it is determined that the tracks have modulation depths of 0.8, 0.7, and 0.9, then the target modulation depth may be further updated in the user model (e.g., reverting to an estimate that a high depth is optimal). In this example, the user model represents estimated effectiveness as a function of modulation depths from 0-1.

In example embodiments, the user model can predict ratings over the modulation characteristic space. For example, if each input track is a point in high-dimensional space (e.g., feature values) each of which has been assigned a color from blue to red (e.g., corresponding to rating values); then the prediction of ratings may be determined by interpolating across known values (e.g., target input tracks) to estimate a heatmap representation of the entire space. In another example, regions of the space can be predicted to contain the highest rating values via linear regression (i.e., if the relationships are simple) or machine learning techniques (e.g., using classifiers, etc.).

In example embodiments, the user model can be distinctive both in terms of the features used (e.g., modulation features relevant to effects on the brain and performance, rather than just musical features relevant to aesthetics) and in terms of the ratings, which can be based on effectiveness to achieve a desired mental state such as, for example, productivity, focus, relaxation, etc. rather than just enjoyment.

In example embodiments, the user model can be treated like a single reference input track if the output to the comparison is a single point in the feature space (e.g., as a “target”) to summarize the user model. This can be done by predicting the point in the feature space that should give the highest ratings and ignoring the rest of the feature space. In this case the process surrounding the user model may not change.

In certain embodiments, a user model may not be required. For example, if multiple reference tracks and ratings are provided as input, the processing device can forgo summarizing them as a model and instead work directly off this provided data. For example, each library track can be scored (e.g., predicted rating) based on its distance from the rated tracks (e.g., weighted by rating; being close to a poorly rated track is bad, etc). This can have a similar outcome as building a user model but does not explicitly require a user model.

In embodiments where only one reference track is used as input, it may be desirable to forgo a user model altogether, and directly compare the reference track to one or more target tracks. This is similar to a user model based only on the one reference track. If the reference track and the one or more target tracks are compared directly, they can be represented in the same dimensional space. Thus, the audio analysis applied to the reference track should result in an output representation that has the same dimensions as the audio analysis that is applied to the one or more target tracks.

At block 130, a set of one or more target audio tracks or a library of target audio tracks can be obtained. The target audio tracks can be, for example, digital audio files retrieved by the processing device from local storage on the processing device or from remote storage on a connected device. In an example, the target audio tracks can be streamed to the processing device from a connected device such as a cloud server for an online music service (e.g., Spotify, Apple Music, etc.). In another example, the target audio tracks may be received by the processing device from an audio input such as a microphone. The sources of the target audio tracks can include, for example, an audio signal, digital music file, musical instrument, or environmental sounds.

In example embodiments, the target audio tracks can be in digital form (e.g., MP3, AAC, WAV, etc.), received as an analog signal, generated by a synthesizer or other signal generator, or recorded by one or more microphones or instrument transducers, etc. The target audio tracks may be embodied as a digital music file (.mp3, .wav, .flac, among others) representing sound pressure values, but can also be a data file read by other software which contains parameters or instructions for sound synthesis, rather than a representation of sound itself. The target audio tracks may be individual instruments in a musical composition, groups of instruments (e.g., bussed outputs), but could also be engineered objects such as frequency subbands (e.g., bass frequencies vs treble frequencies). The content of the target audio tracks may include music, but also non-music such as environmental sounds (wind, water, cafe noise, and so on), or any sound signal such as a microphone input.

In example embodiments, to achieve better brain stimulation, target audio tracks may be selected such that they have a wide (i.e., broadband) spectral audio profile-in other words, the target audio tracks can be selected such that they include many frequency components. For example, the target audio tracks may be selected from music composed from many instruments with timbre that produces overtones across the entire range of human hearing (e.g., 20-20 kHz).

Each target audio track in the set of target audio tracks can include one or more modulation characteristics. Non-limiting examples of these modulation characteristics are modulation depth (i.e., energy/strength of modulation at a particular rate or rates), modulation rate (e.g., dominant modulation rate or rates; i.e., local or global maxima in the modulation spectrum), modulation spectrum (i.e., energy at each modulation rate over a range of rates), joint acoustic and modulation frequency (e.g., modulation rates/spectrum in audio frequency sub bands; e.g., modulation spectrum in the bass region vs. treble region), modulation phase relationships across audio frequency bands, spectro-temporal modulation, metadata such as creator tags and/or labelling indicating any of the above even if not measured directly (e.g., metadata can be added to the audio track at the time of creation from parameters used to make the music, etc.), statistical descriptions of the above; first moment and higher-order moments (e.g,. mean, variance, skewness, kurtosis, etc. of X), time-varying trajectories of the above (i.e., X over time), derivatives of the above; first order and higher order (instantaneous change, acceleration, etc. of X); etc.

At block 140, the desired modulation characteristic values can be compared with the modulation characteristic values of at least one target audio track from the set of target audio tracks. Various techniques can be used for the comparison. In example embodiments, the processing device can take as input either one or more target audio tracks from the set of target audio tracks to compare against the desired modulation characteristic values. If there are many rated reference audio tracks, each reference audio track’s rating value and location in feature space can be considered to define regions in the feature space that are expected to have high ratings (i.e., a user model). This can be framed as a classification problem and can be tackled with any number of methods such as, for example, cluster analysis, decision trees, and/or neural networks.

For example, the difference of 2D modulation spectra (e.g., audio frequency and modulation frequency) between the desired spectrum (as determined by the user model or reference track(s)) and a given target track can be determined by subtraction or by division (% value). Similarly, the difference of 1D modulation spectra (e.g., energy at each modulation frequency across all audio frequencies) can also be determined by subtraction or by division (% value). For example, a 1D modulation spectrum desired by the user model may have normalized power values of 1, 1, 5, 6, 1 at modulation rates of 2 Hz, 4 Hz, 8 Hz, 16 Hz, and 32 Hz, respectively. The 1D modulation spectrum of a first audio track may have normalized power values of 1, 1, 6, 6, 1, at modulation rates of 2 Hz, 4 Hz, 8 Hz, 16 Hz, and 32 Hz, respectively. The 1D modulation spectrum of a second audio track may have normalized power values 2, 3, 6, 10, 1 at modulation rates of 2 Hz, 4 Hz, 8 Hz, 16 Hz, and 32 Hz, respectively. In this example the first audio track, rather than the second audio track, is more similar to the desired spectrum, since the difference of normalized power values is smaller (e.g., 0, 0, 1, 0, 0 versus 1, 2, 1, 4, 0). Similarity in time-averaged properties, versus similarity over time (i.e., average vs. trajectories) can also be used for the comparison.

At block 150, a target audio track can be selected from the set of at least one target audio tracks based on the comparing, wherein the modulation characteristic values of the target audio track best match the desired modulation characteristic values. If the comparing is defined as a function over the space, this may be done by selecting the target audio track with the highest predicted efficacy under a user model (if used). If the model is defined by a single ‘best’ point or region in the space rather than a function, then determining the best match can be done by finding the closest track (euclidean distance in multiple dimensions). For example, if the model dimensions are modulation depth at 4 Hz and modulation depth at 12 Hz, and if the desired (highest predicted efficacy) under the user model is at a depth of 3 and 7 for 4 Hz and 12 Hz respectively, then an audio track with depths of 4 and 8 at 4 Hz and 12 Hz respectively, would have a calculated euclidean distance from the target of sqrt((7-3)^2 + (8-4)^2)=5.67. This value would be compared against the distance value from other tracks to select the closest target track to the desired modulation characteristic value(s).

In some embodiments, the target audio track may be modified as subsequently described in block 360. For example, if the user provides input that they have ADHD, then the user model may indicate that the target audio track should have a spectral slope (treble-bass balance) of 0.6. If, however, the library of target audio tracks contains only audio tracks with spectral slope between 0.1-0.4, then the target audio track with the highest slope (closest to 0.6) may be selected, and further modified to have spectral slope of 0.6. The modification may be done, for example, by low pass filtering.

At block 160, the selected target audio track can be played via one or more audio drivers of one or more playback devices, such as, for example, a smart speaker, a mobile device, a computer/laptop, an ipad, and the like. In one example, the processing device is the same device as the playback device, and the target audio track can be played via audio drivers on the processing device itself. In another example, the processing device can transmit the target audio track (e.g., as a digital file over a data network) to a playback device for playback. In another example, the target audio track can be played on the processing device as well as other playback devices. In another example, the target audio track can be stored (e.g., in a playlist) for future playback.

In example embodiments, the selection of a target audio track for playback at block 160 responsive to the user-associated data at block 110 can be based on a measure of the effectiveness of the user reaching a target mental state with one or more previously played reference audio tracks; these could be tracks included in the library of target tracks but are defined as reference tracks once used as input to the system along with user-associated data (e.g., ratings of those tracks). For example, a target audio track can be selected based on effectiveness rating of previously played reference audio track(s) by the user and modulation characteristics of one or more target audio tracks. This is different from known technology that selects audio tracks based on aesthetic rating and/or music parameters. Another non-limiting example can be that a second audio track is selected for playback based on a first track by implicitly determining (e.g., based on user history, or user devices such as Oura ring that recognizes sleep patterns, etc.,) if the first track is effective. In such a scenario, knowledge of a desired mental state may not be required.

### B. Audio Parameters and Modulation Characteristics

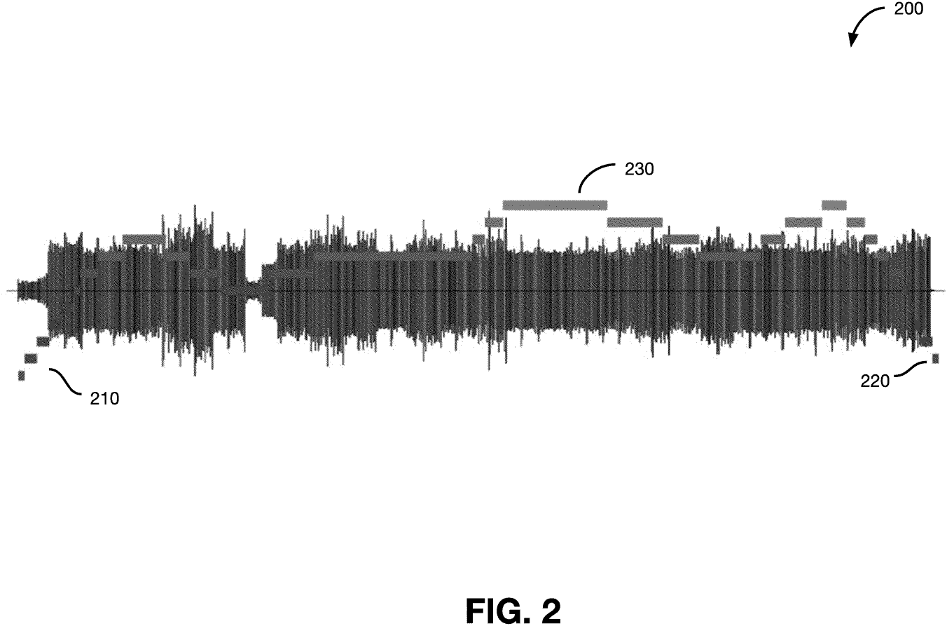

FIG. 2 shows a waveform of an example audio track 205 overlaid with its analyzed modulation depth trajectory according to an embodiment of the present disclosure. In this example, the modulation depth 200 starts low 210, ends low 220, and varies over time during the body of the audio content, with a high plateau 230 starting about halfway through the track. This pattern may be beneficial to provide a targeted mental state such as focus, mediation, relaxation, etc.

As shown in FIG. 2, the audio content (e.g., music, sound effects, or other content) is different from the overlaid modulation. Aspects of the music (sometimes referred to herein as “audio parameters”) such as tempo, RMS (root mean square) energy in the audio signal, spectral brightness, timbre, harmonicity, and so on are different from the aspects of the overlaid modulation (sometimes referred to herein as “modulation characteristics”) such as the modulation depth, modulation frequency, modulation rate, and so on.

Audio parameters and modulation characteristics are described further herein in the context of their use in both selecting and/or modifying media items for playback in connection with the disclosed embodiments.

### C. Selecting Media Items for Playback Based on Desired Modulation Characteristics and Desired Audio Parameters

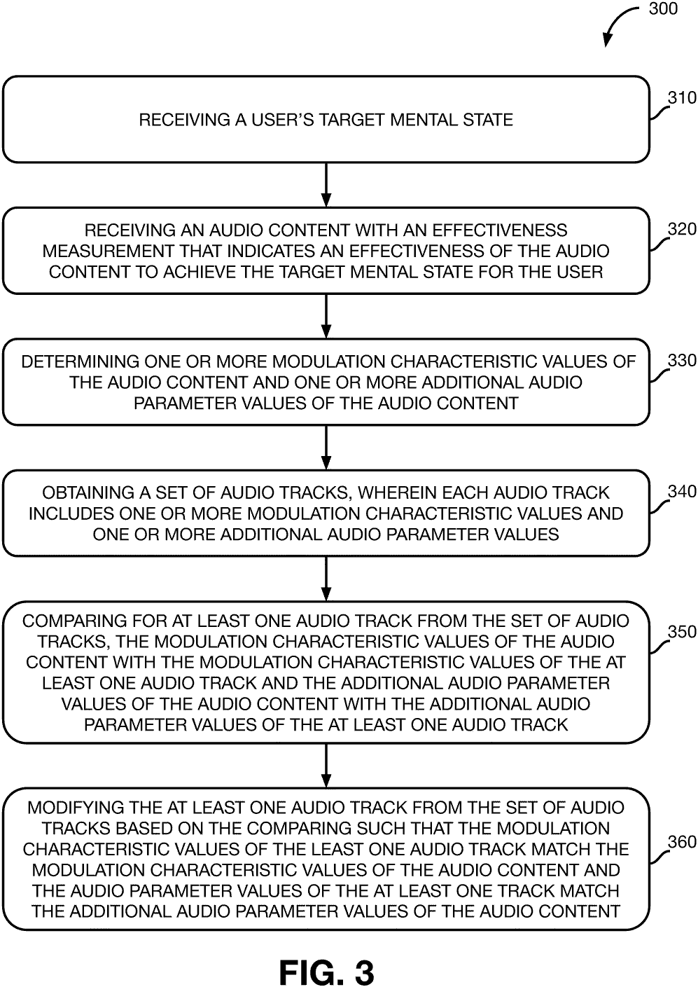

FIG. 3 illustrates an example method 300 performed by a processing device (e.g., smartphone, computer, smart speaker, etc.) according to an example embodiment of the present disclosure. According to example embodiments of the present disclosure, method 300 can be performed by the same processing device that performs method 100. Alternatively, method 300 can be performed by a different processing device (e.g., smartphone, computer, etc.). The method 300 may include one or more operations, functions, or actions as illustrated in one or more of blocks 310-360. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation.

At block 310, a user’s target mental state can be received. Certain aspects of block 310 have been previously described with respect to method 100. Non-limiting examples of a user’s target mental state can include focus, relax, sleep, and meditate. Each of these example desired mental states can be further distinguished by a target activity and duration. For example, focus can be distinguished by deep work, creative flow, study and read, light work, etc.; relax can be distinguished by chill, recharge, destress, unwind, etc.; sleep can be distinguished by deep sleep, guided sleep, sleep and wake, wind down, etc.; and meditate can be distinguished by unguided and guided. The duration of the mental state may be specified, for example, by a time duration (e.g., minutes, hours, etc.), or a duration triggered by an event (e.g., waking, etc.). The indication may be received via a user interface on a processing device such as, for example, through an interface on the Brain.fm™ application executing on an iPhone™ or Android™ device. Alternatively, and/or additionally, the indication may be received over a network from a different processing device.

At block 320, a reference audio content with an effectiveness measurement can be received. Certain aspects of block 310 have been previously described with respect to method 100. The effectiveness measurement may indicate an effectiveness of the reference audio content to achieve the target mental state for the user. The effectiveness measurement can be implicitly defined as effective by the user merely providing a reference audio content to the system.

At block 330, one or more modulation characteristic values of the reference audio content and one or more additional audio parameter values of the reference audio content can be determined. Certain aspects of block 330 have been previously described with respect to method 100. Non-limiting examples of audio parameters may include tempo; RMS (root mean square energy in signal); loudness; event density; spectrum/spectral/envelope/brightness; temporal envelope; cepstrum (e.g., spectrum of spectrum); chromagram (e.g., what pitches dominate); flux (e.g., change over time); autocorrelation; amplitude modulation spectrum (e.g., how energy distributed over temporal modulation rates); spectral modulation spectrum (e.g., how energy distributed over spectral modulation rates); attack and decay (e.g., rise/fall time of audio events); roughness (e.g., more spectral peaks close together is rougher); harmonicity/inharmonicity (i.e., related to roughness but calculated differently); and/or zero crossings (i.e., sparseness).

Various techniques can be used to identify additional audio parameter values associated with audio content. Non-limiting examples of such techniques can include multi-timescale analysis of features (e.g., different window lengths); analysis of features over time; broadband or within frequency subbands (i.e. after filtering); and/or second order relationships (e.g., flux of cepstrum, autocorrelation of flux). Additionally, or alternatively, additional audio parameter values may be identified in a metadata field associated with audio content.

At block 340, a set of one or more target audio tracks can be obtained such that each target audio track includes one or more modulation characteristic values and one or more additional audio parameter values. Certain aspects of block 340 have been previously described with respect to method 100. In some embodiments, the obtained set of one or more target audio tracks can be based on, for example, a target mental state of the user, an aesthetic perception of whether a reference audio track sounds good, and/or unique properties of a reference audio track relative to others (i.e., distinctiveness).

At block 350, for at least one target audio track from the set of one or more target audio tracks, the one or more modulation characteristic values of the reference audio content can be compared with the one or more modulation characteristic values of the at least one target audio track and the additional one or more audio parameter values of the reference audio content can be compared with the one or more additional audio parameter values of the at least one target audio track. Certain aspects of block 350 have been previously described with respect to method 100.

At block 360, the at least one target audio track from the set of target audio tracks can be modified based on the comparing such that the one or more modulation characteristic values of the at least one target audio track substantially matches the one or more modulation characteristic values of the reference audio content and the one or more audio parameter values of the at least one target audio track substantially matches the one or more additional audio parameter values of the reference audio content. For example, if a user with ADHD prefers listening to a particular pop song to focus, then the modulation characteristics of that pop song can be modified (e.g. change the modulation depth to 12-20 Hz) based on the target “focus” mental state for the user. In one embodiment where the selected target audio track is sufficiently similar in the comparing, block 360 can be omitted.

In an example embodiment, the processing device may select a subsequent target audio track from the set of target audio tracks based on the comparing (as described by block 350) such that the modulation characteristic values of a beginning portion of the subsequent target audio track aligns in a predetermined manner with an end portion of the reference audio track. In this case the processing device may use the heads and tails of audio tracks instead of the entire track. The processing device may then sequentially combine, or chain, the reference audio track and the subsequent selected target audio track. When the audio tracks are combined, the start and end regions (e.g., where modulation depth is low) can be removed to avoid a dip in modulation depth (e.g., potentially disrupting the effect of modulation). The resulting combination of audio tracks can have more consistent modulation depth and may be valuable to the user by maintaining the desired mental state.

In one embodiment, heads and tails of audio tracks can be used to chain audio tracks together to create a playlist with modulation characteristics and/or other audio characteristics (e.g., as described above) that are smooth and continuous across track changes. In another embodiment, audio tracks can be chained based on contrasting (i.e., maximally different) modulation characteristics and/or other audio characteristics. In yet another embodiment, target audio tracks can be chained based on a combination of both contrasting and similar modulation characteristics and/or other audio characteristics.

In an example embodiment, an acoustic analysis can be performed on the modified target audio content. The analysis can include determining a distance, in measurement space (i.e., the space of measured modulation characteristics and/or audio characteristics), between the modified target audio content and a reference audio content. The determined distance can define a cost function in the space of modifiable parameters. The cost function can then be evaluated by applying optimization techniques, which can involve selecting multiple sample points in the parameter space, modifying the audio, and finding the distance in measurement space at each sampled point in the parameter space. The target audio content can also be modified repeatedly until a global minimum in the cost function can be adequately estimated. The target audio content can then be further modified according to the estimated optimum parameters or the modified target audio can be retrieved if already close to this optimum.

Alternatively, in an example embodiment, a mapping can be provided that translates between parameter space and measurement space such that a movement in parameter space would result in a known movement in measurement space. Similarly, the parameter space and measurement space can be chosen to be heavily interrelated, e.g., if the parameter space is the depth and rate of broadband tremolo, and the measurement space is the depth and rate of broadband modulation. In these cases, the optimization over a latent function (i.e., minimization of the cost function defined by the reference-target difference in measurement space at each point in the target-modification parameter space) is not required since the location of the modified target in measurement space can be estimated directly by the change in parameters during modification.

In example embodiments, one or more target audio tracks can be modified to move toward a particular location in a significant feature space, e.g., modulation depth and rate. The parameter space, e.g., the many knobs that can be turned to modify the audio, may not be the same as the measurement space (feature space), which relates the music to effects on the brain.

In example embodiments, the set of one or more target audio tracks can include a single target audio track only. In such a case, that single target audio track can be modified along various dimensions as described with respect to block 360. The modulation characteristic of the single target audio track can be modified based on user inputs (e.g., prescribed modulation characteristics values). For example, if a user completes a survey that shows they have ADHD, and it is known that they will benefit from a particular modulation depth at 12-20 Hz rate, then the user can select the closest target audio track from a library of target audio tracks. The selected target audio track may still not be ideal. In such a case, the target audio track can be modified to have the desired modulation characteristics values.

### D. Identifying Media Items Based on Similarity to a Reference Media Item

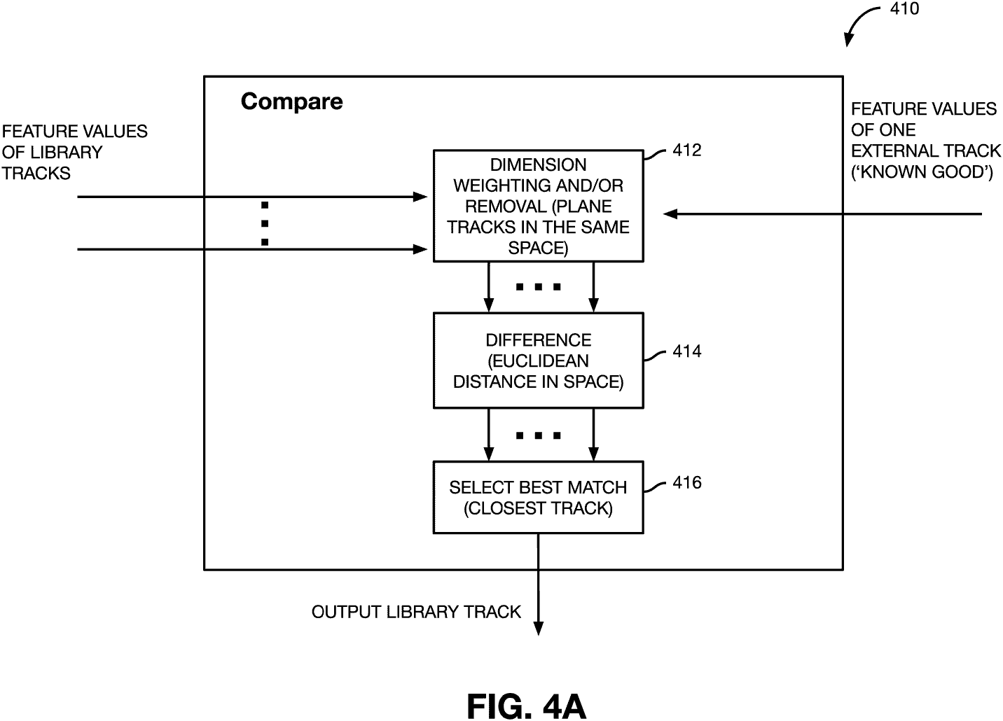

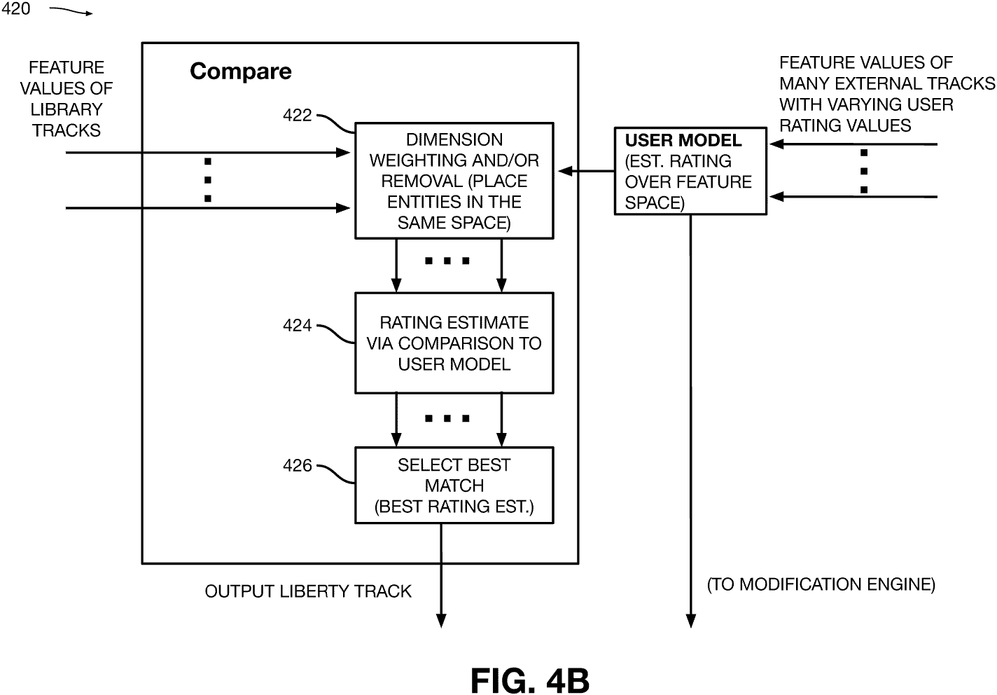

FIG. 4A provides an example illustration of the comparison process flow 410 as previously described with respect to block 350 of method 300, when using a single reference track rather than a user model The process flows 410 may include one or more operations, functions, or actions as illustrated in one or more of blocks 412-416. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation.

As shown in FIG. 4A, feature values of the target audio tracks 411 and feature values of the reference audio tracks 413 are input to the comparison block 410. In this example, a dimension weighting and/or removal block 412 takes these input values and determines which feature dimensions (if any) should be reweighted or removed to establish a feature space that is common to the target and reference tracks, and is most relevant to the user’s desired mental state. For example, if only the reference track(s) have an analyzed dimension of ‘modulation phase’, but the target track does not, this dimension could be removed prior to comparison. Similarly, if the user is known to want a mental state of Focus, but analyzed dimensions exist that are known to be irrelevant to focus, these dimensions could be removed by process 412/422 prior to comparison in blocks 414/424.

A difference block 414 takes the output of dimension weighting and/or removal block 412 and determines the difference (e.g., in Euclidean distance space) between reference and targets. In an example embodiment, modification 360 may not move a target audio track arbitrarily in feature space; there are limited directions and distances based on audio processing techniques, the target audio track to be modified, and other constraints. For example, consider one rated audio track T and two audio tracks A and B in a library of audio tracks. In this example, it may be the case that T-A > T-B (i.e., the difference between T and A is greater than the difference between T and B; so, B seems best), but the distance T-B cannot be traversed by available audio modification techniques, whereas T-A can be traversed by available audio modification techniques. A practical example may be if T and A differ greatly in brightness (e.g., spectral tilt), which can be modified by filtering/EQ without impacting other dimensions, whereas T and B differ in the phase of modulation across frequency bands, which is not easy to modify (e.g., may require removing instruments, etc). In this case, B may be selected as being more similar in the process 410 (method 100), but A may be selected for modification in the process 420 (method 300). In block 416, the best match is selected and the target audio track is output 415.

### E. Identifying Media Items Based on Similarity to a User Model

FIG. 4B provides an example illustration of the comparison process flow 420 as previously described with respect to block 350 of method 300, when a user model is used. The process flows 410, 420 may include one or more operations, functions, or actions as illustrated in one or more of blocks 412-426. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation.

Unlike in method 100, method 300 may consider how modification can move a target audio track through the feature space. Thus, process 422, in addition to establishing a common feature space between target and reference as in process 412, may reweight the feature space to reflect the possibilities of movement by audio modification. For example, a dimension corresponding to brightness (e.g., which may be easy to manipulate) may be compressed such that a difference in that dimension is down-weighted in the comparison. This allows process 420 to find a target audio track which can be modified to be the best match 426 (optimal features under the user model), whereas process 410 aims to find a target audio track that substantially matches the desired modulation characteristics 416 (closest match to a reference track known to have desirable characteristics).

### F. Example Computing Devices for Practicing Disclosed Embodiments

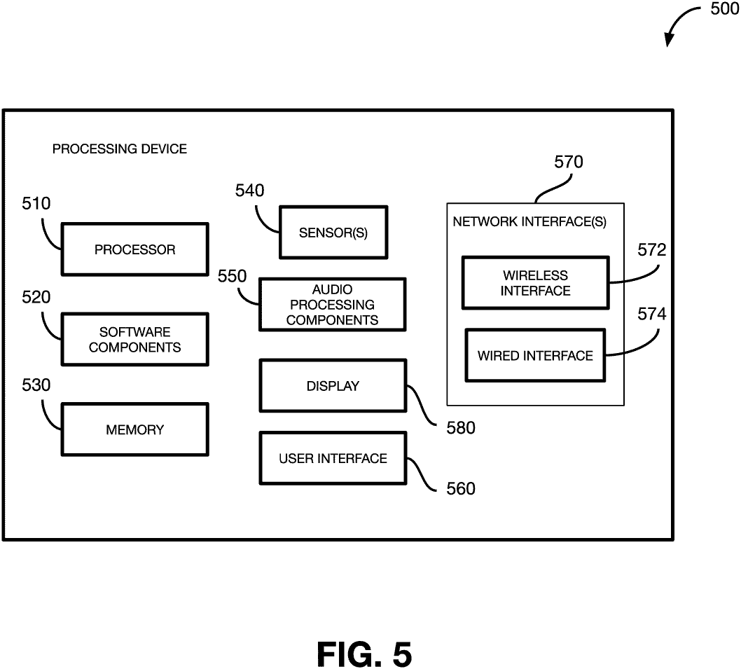

FIG. 5 shows a functional block diagram of an example processing device 500 that can implement the previously described methods 100 and 300, process flows 410 and 520, and the closed loop system embodiments described below. The processing device 500 includes one or more processors 510, software components 520, memory 530, one or more sensor inputs 540, audio processing components (e.g. audio input) 550, a user interface 560, a network interface 570 including wireless interface(s) 572 and/or wired interface(s) 574, and a display 580. The processing device may further optionally include audio amplifier(s) and speaker(s) for audio playback. In one case, the processing device 500 may not include the speaker(s), but rather a speaker interface for connecting the processing device to external speakers. In another case, the processing device 500 may include neither the speaker(s) nor the audio amplifier(s), but rather an audio interface for connecting the processing device 500 to an external audio amplifier or audio-visual playback device.

In some examples, the one or more processors 510 include one or more clock-driven computing components configured to process input data according to instructions stored in the memory 530. The memory 530 may be a tangible, non-transitory computer-readable medium configured to store instructions executable by the one or more processors 510. For instance, the memory 530 may be data storage that can be loaded with one or more of the software components 520 executable by the one or more processors 510 to achieve certain functions. In one example, the functions may involve the processing device 500 retrieving audio data from an audio source or another processing device. In another example, the functions may involve the processing device 500 sending audio data to another device or a playback device on a network.

The audio processing components 550 may include one or more digital-to-analog converters (DAC), an audio preprocessing component, an audio enhancement component or a digital signal processor (DSP), and so on. In one embodiment, one or more of the audio processing components 550 may be a subcomponent of the one or more processors 510. In one example, audio content may be processed and/or intentionally altered by the audio processing components 550 to produce audio signals. The produced audio signals may be further processed and/or provided to an amplifier for playback.

The network interface 570 may be configured to facilitate a data flow between the processing device 500 and one or more other devices on a data network, including but not limited to data to/from other processing devices, playback devices, storage devices, and the like. As such, the processing device 500 may be configured to transmit and receive audio content over the data network from one or more other devices in communication with the processing device 500, network devices within a local area network (LAN), or audio content sources over a wide area network (WAN) such as the Internet. The processing device 500 may also be configured to transmit and receive sensor input over the data network from one or more other devices in communication with the processing device 500, network devices within a LAN or over a WAN such as the Internet. The processing device 500 may also be configured to transmit and receive audio processing information such as, for example, a sensor-modulation-characteristic table over the data network from one or more other devices in communication with the processing device 500, network devices within a LAN or over a WAN such as the Internet.

As shown in FIG. 5, the network interface 570 may include wireless interface(s) 572 and wired interface(s) 574. The wireless interface(s) 572 may provide network interface functions for the processing device 500 to wirelessly communicate with other devices in accordance with a communication protocol (e.g., any wireless standard including the IEEE 802.11a/b/g/n/ac, 802.15, 4G/5G mobile communication standards, and so on). The wired interface(s) 574 may provide network interface functions for the processing device 500 to communicate over a wired connection with other devices in accordance with a communication protocol (e.g., IEEE802.3). While the network interface 570 shown in FIG. 5 includes both wireless interface(s) 572 and wired interface(s) 574, the network interface 570 may in some embodiments include only wireless interface(s) or only wired interface(s).

The processing device may include one or more sensor(s) 540. The sensors 540 may include, for example, inertial sensors (e.g., accelerometer, gyrometer, and magnetometer), a microphone, a camera, or a physiological sensor such as, for example, a sensor that measures heart rate, blood pressure, body temperature, EEG, MEG, Near infrared (fNIRS), or bodily fluid. In some example embodiments, the sensor may correspond to a measure of user activity on a device such as, for example, a smart phone, computer, tablet, or the like.

The user interface 560 and display 580 can be configured to facilitate user access and control of the processing device. Example user interface 560 include a keyboard, touchscreen on a display, navigation device (e.g., mouse), etc.

### G. Example Computing Systems for Practicing Disclosed Embodiments

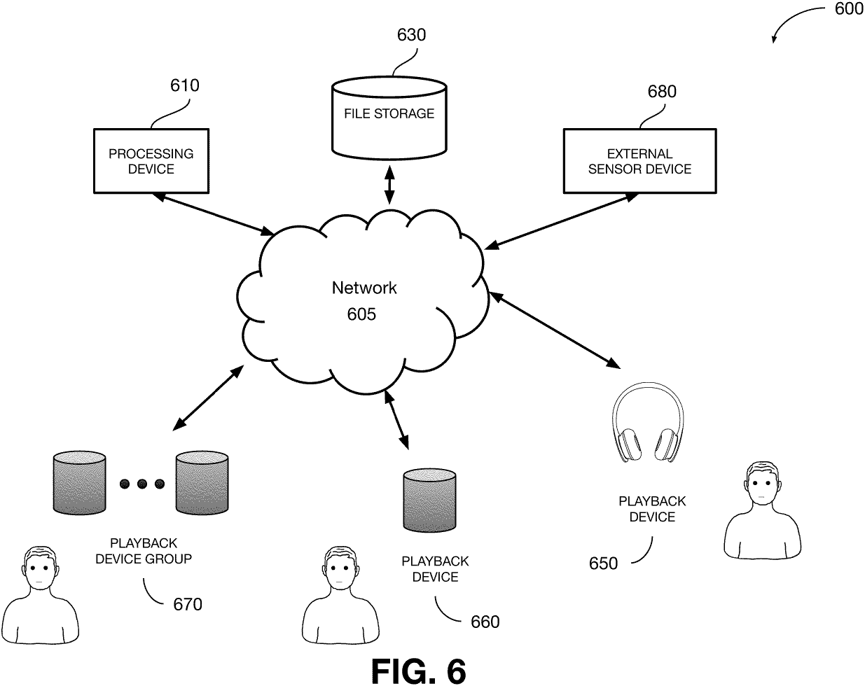

Aspects of the present disclosure may exist in part or wholly in, distributed across, or duplicated across one or more physical devices. FIG. 6 illustrates one such example system 600 in which the present invention may be practiced. The system 600 illustrates several devices (e.g., processing device 610, audio processing device 620, file storage 630, playback device 650, 660, and playback device group 670) interconnected via a data network 605. Although the devices are shown individually, the devices may be combined into fewer devices, separated into additional devices, and/or removed based upon an implementation. The data network 605 may be a wired network, a wireless network, or a combination of both.

In some example embodiments, the system 600 can include an audio processing device that can perform various functions, including but not limited to audio processing. In an example embodiment, the system 600 can include a processing device 610 that can perform various functions, including but not limited to, aiding the processing by the audio processing device 620. In an example embodiment, the processing devices 610 can be implemented on a machine such as the previously described system 500.

In an example embodiment, the system 600 can include a storage 630 that is connected to various components of the system 600 via a network 605. The connection can also be wired (not shown). The storage 630 can be configured to store data/information generated or utilized by the presently described techniques. For example, the storage 630 can store the set of one or more target audio tracks, as previously discussed with respect to the steps 130 and 340. The storage 630 can also store the audio track in the step 160.

In an example embodiment, the system 600 can include one or more playback devices 650, 660 or a group of playback devices 670 (e.g. playback devices, speakers, mobile devices, etc.). These devices can be used to playback the audio output, as previously described in the step 180. In some example embodiments, a playback device may include some or all of the functionality of the processing device 610, the audio processing device 620, and/or the file storage 630. As described previously, a sensor can be based on the audio processing device 620 or it can be an external sensor device 680 and data from the sensor can be transferred to the audio processing device 620.

### H. Monitoring and Regulating Mental States Within a Multi-Dimensional Mental State Model

Embodiments disclosed herein may be used for a closed-loop system to regulate mental states with audio. For example, biological attributes (e.g., heart rate) associated with the user may be monitored using sensors (e.g., earbud biosensors) and these measurements may be used to dynamically select and/or modify the audio for the user to reach a desired mental state. The system may allow for a continuous or near continuous monitoring of the user’s state and dynamically selecting/modifying the audio based on the monitoring.

The system may address various user requirements not met by conventional systems. For instance, although biological sensors may be used to identify a user’s current mental state, conventional systems may not be able to predictably move a user’s current mental state to another mental state using conventional audio systems. For example, a system may provide audio content that generally regulates a user’s mental state without actively monitoring the user’s mental state and/or dynamically modifying the audio content to adjust for the user’s changing mental state. Furthermore, conventional systems do not provide mechanisms to (i) display a user’s existing mental state; (ii)allow users to select a desired change in their mental state, whereby the selection is used to initiate audio content playback that is designed to transition the user’s mental state from their existing mental state to a desired mental state; and (iii)provide feedback on a user interface showing the change in the user’s mental state over time.

The system may address these and other requirements not met by conventional systems and may provide additional benefits as well. The systems may use one or more sensors to measure biological attributes of a user to monitor their mental state. For instance, a sensor such as an earbud biosensor (e.g., a sensor embedded in an earbud that is used for audio playback) may measure PPG (photoplethesmogram) data, which may include heart rate, heart rate variance, blood pressure, and the like. Similarly, an accelerometer (e.g., within the earbud or other wearable) may measure the movement data. A temperature sensor, which may also be within the earbud or other wearable, may measure the user’s body temperature. Other measurements may also be taken, e.g., a photo, a series of photos, or video of the user’s face, etc.. One or more of the aforementioned measurements may be used to determine the user’s mental state (e.g., level of stress based on one or more stress metrics).

The measurements can then be used to dynamically select and/or modify the audio for a user. The dynamic selection/modification may be one-dimensional, e.g., to control the stress levels (generally de-stress). The dynamic selection/modification may be multi-dimensional, e.g., to control valence (emotional state of the user, ranging from serious to cheerful) and arousal (energy level of the user, ranging from calm to energized). As described herein, the two-dimensional (or multi-dimensional) control by audio selection/modification may be indicated as a movement of a user’s mental state through a multi-dimensional space. For instance, the user’s mental state may move (or maintain a location) in a valence-arousal space based on the audio the user is listening to. The use of mental state space and vectors causing the movement within the space is just for explanation and not intended to be limiting. The selections/modifications described above may be based on mapping between the measurement data (and/or corresponding position in the multi-dimensional space) to audio features—where the mapping relationship may be learned by a model and/or developed through research. As described herein, an audio selection may be made from a library of audio tracks; and a modification may be made within the same audio track (e.g., same song) and/or the determination of a new audio track.

In one example of two-dimensional control, the operation of the closed loop system may be described in terms of moving a user in a valence-arousal space (one example of a multi-dimensional mental state space) using audio, monitoring the movement within the mental state space using sensors, and dynamically selecting/modifying the audio to control/adjust the movement within the mental state space. In operation, the current position within the valence-arousal space (or other mental state space) corresponds to the user’s current mental state. The user’s current mental state (and the user’s mental state over time) can be determined via user inputs and/or sensor inputs relating to the user’s mental state.

For example, in some embodiments, a current location in a mental state space (corresponding to the user’s current mental state) can be determined by (i) one or more sensor inputs from wearable biosensors (e.g., a smart watch, smart ring, other wearable biosensors or wearable devices incorporating biosensors), (ii) direct user input, e.g., an indication of the user’s current mental state received from the user via a user input, and/or (ii) inference/estimation based on external data, e.g., current weather or time of day, a keystroke history from a computing device associated with the user, the user’s GPS location, or other external data sources.

In some embodiments, the user’s determined mental state (and the user’s mental state over time) is mapped to a position (or a series of positions to form a trajectory) within a multi-dimensional state space to facilitate monitoring and tracking of the user’s mental state over time.

Some embodiments disclosed herein include mapping the user’s mental states to a multi-dimensional mental state space and selecting and/or modifying (and then playing) media content to move the user’s mental state in a desired trajectory through the multi-dimensional mental state space. In such embodiments, and as described further herein, different media content may have different attributes (e.g., different audio parameters and/or different modulation characteristics) that are designed to cause a change in the user’s mental state. In some embodiments, sensor data is used to track the user’s mental state as the user is experiencing (e.g., listening to, watching, or otherwise experiencing) the media item.

Based on the tracking of the user’s mental state, either (i) the media item can be modified (e.g., by changing one or more audio parameters and/or one or more modulation characteristics) during playback, or (ii) a new media item can be selected for playback. The modified media item (or the new media item, depending on the implementation) can then be played. By monitoring the user’s mental state and dynamically changing the media item being played (e.g., by modifying the media item or playing a different media item), disclosed embodiments are able to control a trajectory of the user’s mental state within the multi-dimensional mental state space to help the user achieve a desired target mental state.

The movement data about the multi-dimensional mental state space (reflecting changes in the user’s mental state over time) may be captured, as described above, using sensor data (e.g., PPG data). Machine learning models may be used to learn the patterns in the sensor data that correspond to the how the user’s mental state is changing within the multi-dimensional mental state space. The sensor data (along with learned patterns) may be added to other data (e.g., data from earbud sensors, facial recognition using a phone camera) to estimate the user’s location within the multi-dimensional mental state space. The user’s location within the multi-dimensional mental state space (corresponding to the user’s mental state within the multi-dimensional mental state space) may indicate, as described above, how cheerful or serious the user is feeling (emotional valence) and how calm or energized the user is feeling (arousal). The multi-dimensional mental state space may also be composed of any other dimensions of mental states, for example: sleepy to awake (wakefulness), creatively-blocked to inspired (creativity), unmotivated to motivated (motivation), and so on. As mentioned earlier, this location within the multi-dimensional mental state space may be influenced by the audio the user is listening to, and modifying the audio may therefore allow the user to change his or her mental state, which corresponds to movement within the multi-dimensional mental state space. Concretely, a particular type of music may be associated with cheerfulness, and when this type of music is selected and played, the user’s direction of movement in mental state space may be away from seriousness and towards cheerfulness.

Some embodiments include analyzing audio tracks (e.g., through machine learning techniques, clinical observation, and/or user feedback either individually or groupwise) as to whether and the extent to which individual audio tracks tend to cause changes in a user’s mental state. Changing the user’s mental state can be mapped to a trajectory within the multi-dimensional mental state space. Some embodiments include maintaining a library of audio tracks, where each track in the library has a corresponding expected trajectory within the multi-dimensional mental state space. This trajectory is referred to herein as the expected trajectory because it reflects the mental state trajectory that the audio track is expected to cause the listener to experience.

As an example, an audio track may be analyzed to determine a mental state vector decomposition such that the vector may represent the projected movement within the multi-dimensional mental state space, i.e., the audio track’s expected trajectory. The association between the audio track and the audio track’s corresponding expected trajectory may be determined by, for example, training a model on the particular users and/or other users, and/or hand-engineering the relationship based on research (e.g., to extract hand-crafted features) or other knowledge. A multidimensional vector may be determined as a multidimensional object, or may be the composition of single-dimensional vectors (i.e. the effect of the audio may be determined in each dimension independently and summed to produce a multidimensional vector). In some embodiments, if a model is trained (and/or is developed another way) for a group of users, the model may be fine-tuned, for example, by further training and/or further research, for a particular user.

Based on the aforementioned and/or similar analysis, an audio track (or other media item) can be associated with a direction of a movement in the multi-dimensional mental state space and/or a force vector (having both direction and magnitude). The direction of movement and/or force vector is sometimes referred to herein as a trajectory within a multi-dimensional mental state space. The direction of movement refers to, for example, movement along a gradient between extremes of mental states, such as a transition from one (or more) mental states to another one (or more) mental states. For example, the direction may correspond to moving in a direction from being relatively relaxed toward being more excited than relaxed, or from relatively energized and serious to more cheerful and calm. The magnitude may represent, for example, the reliability of the expected effect, the speed of the expected effect, the start-to-end distance of the expected effect, and/or some other value. For example, in some scenarios, the magnitude may characterize how strongly the audio track is expected to move the user’s mental state in a certain direction within the multi-dimensional mental state space and/or the distance of movement in the space. Another form of audio and mental state space association may include an association of the audio with a location in the space that may draw the listener toward the location.

### I. Example Graphical User Interfaces Showing Mental State Spaces and Trajectories

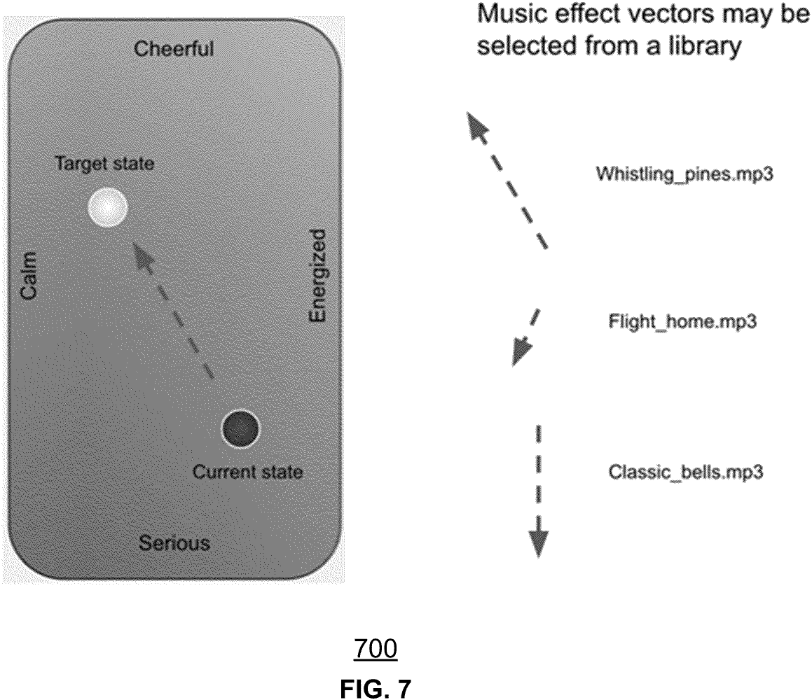

FIG. 7 depicts an example graphical user interface 700 with example vectors (sometimes referred to herein as trajectories) representing the effect of music within a mental state space. The corresponding vectors / trajectories are shown for three example pieces of music (i) whistling pine, (ii) flight home, and (iii) classic bells. Furthermore, the current state (corresponding to a user’s current mental state) and target state (corresponding to a desired target mental state) are shown in the multi-dimensional mental state space (with the vertical dimension being valence and the horizontal dimension being arousal). As shown, the whistling pine piece may provide an effect that may more likely move the user from the current state to the target state. The system may therefore select the whistling pine piece for the shown scenario rather than the flight home or classic bells tracks because the expected trajectory of the whistling pines track better approximates the desired trajectory from the user’s current state to the user’s target state than the expected trajectories of the flight home or classic bells tracks.

In addition to the selection of an audio track (e.g., selecting a particular piece of music), the selected audio itself may be modified, as described above and below, to change the effect that the audio track is likely to have on the user’s mental state. In this way, the audio track can be modified to change the audio track’s expected trajectory. Some example parameters that can be modified include modulation, brightness, volume level, etc. For example, a media item (e.g., an audio music track with or without modulation characteristics) may be modified over time to shift the user along a certain trajectory within the multi-dimensional mental state space. For example, a user may “overshoot” his/her target, and this overshooting may be detected in the multi-dimensional mental state space (e.g., by monitoring the sensors) and the audio being played itself may be modified (e.g., modifying its modulation depth) to compensate for the overshoot, instead of stopping or changing the audio track.

The closed loop system may further provide dynamic flexibility between modifying the audio track being played or selecting a new audio track. For example, a popular audio track, which may shift most users rightward by distance X in the multi-dimensional mental state space, may shift a particular user by distance X/2. Once this sub-optimal shift is detected by the one or more sensors, the audio track itself may be modified to produce a greater rightward shift for that particular user. Alternatively, the audio track may be terminated, and a new track may be selected and played instead of dynamically modifying the audio track that was being played first. This flexibility allows for desired movement within the multi-dimensional mental state space by any combination of track modifications (i.e., modifying one or more audio parameters of the track and/or modifying one or more modulation characteristics of the track) and track changes (e.g., transitioning from playback of a first track to playback of a second track).

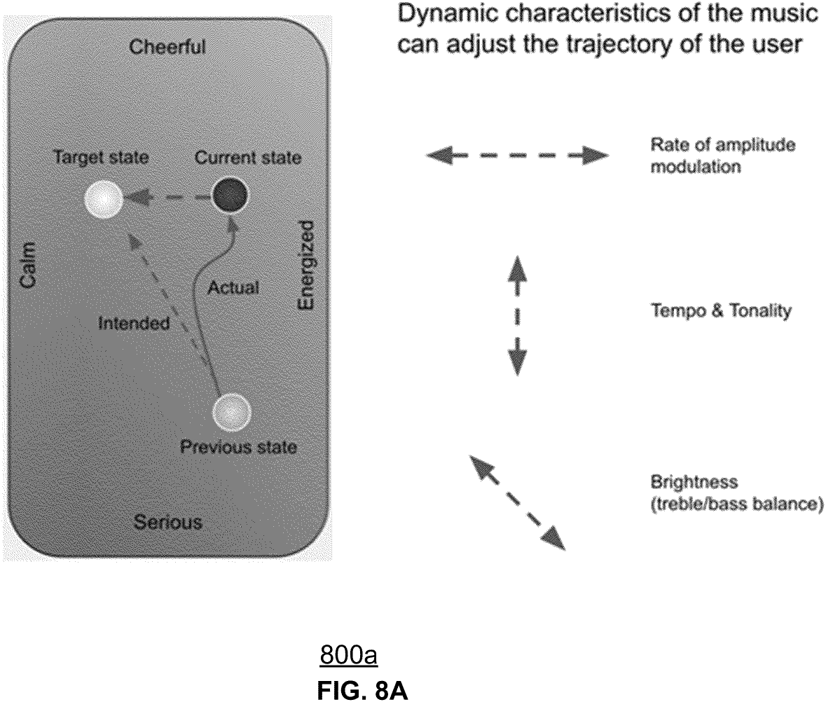

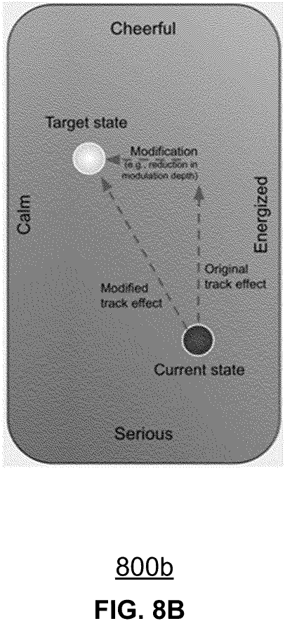

For example, FIG. 8A depicts another graphical user interface 800a showing a comparison of and intended movement (sometimes referred to as a desired trajectory) versus an actual movement (sometimes referred to as a measured trajectory) within the multi-dimensional mental state space. As shown, the intended movement towards the target state from a previous state (i.e., the desired trajectory) was not necessarily achieved—there is a deviation from the intended path within the multi-dimensional mental state space that takes the user to the current state (corresponding to the user’s current mental state), which is rightward of the target state (corresponding to a desired target mental state for the user). Parameters of the audio track may be dynamically modified such that the user may move from the current state to the target state. As shown, the example parameter candidates include (i) modulation characteristics, such as the rate of amplitude modulation, the intensity of the modulation, and so on, and (ii) audio parameters of the audio track, such as tempo and tonality, and/or brightness (indicating a bass/treble balance). For example, the amplitude modulation frequency for a focus state may be 12-20 Hz and the amplitude modulation frequency for a sleep state may be 0.25 -1 Hz. Tempo and movement in emotional valence may be associated with each other, e.g., faster music may be associated with more cheerfulness, ceteris paribus. A higher brightness (e.g., increased treble and lowered bass) is generally associated with calmness and cheerfulness, ceteris paribus.

FIG. 8B depicts an interface 800b showing how modifying the audio track, e.g., by modifying one or more audio parameters and/or modulation characteristics as described above (and below), and then playing the modified audio track, may help to move a user to the user’s desired target mental state, compared to playing the original audio track without the modifications. As shown, the expected trajectory of the original track took the user to the right of the desired target state (as shown in FIG. 8A), whereas the modified trajectory of the audio track takes the user to the target state. Therefore, modifying one or more aspects of the audio track modifies the original trajectory of the audio track (resulting in a modified trajectory). In operation, modifying the expected trajectory of an audio track in this manner, and then playing the modified audio track, enables more fine-tuned control over the trajectory of the user’s mental state within the multi-dimensional mental state space as compared to using static, unmodified audio tracks from the library of audio tracks with predetermined effect vectors. The modification therefore may allow for more granular ways to control the user’s mental state trajectory between different locations within the multi-dimensional mental state space. The users may also be able to listen to familiar favorites with different flavors depending on what they need at the moment. These modifications may be done in real time, e.g., based on the closed loop feedback from the sensors; and/or at the beginning of the playback based on the current state and the desired state of the user. These modifications allow adjustment of the user’s mental state trajectory without needing to play an entirely different audio track, which may be jarring or otherwise undesirable.

The closed loop system may also provide interfaces to users to interact with the multi-dimensional mental state spaces . The above-described FIGS. 7, 8A, and 8B show some example graphical user interfaces 700, 800a, and 800b. Additional graphical user interfaces described below may allow the users to see their estimated locations (e.g., a location or a region within the multi-dimensional mental state space).

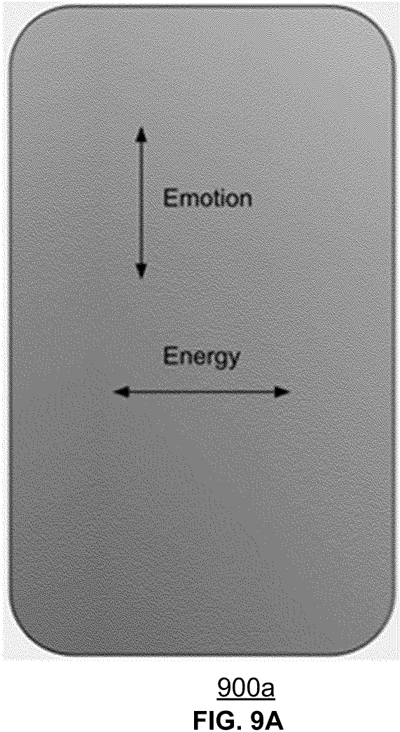

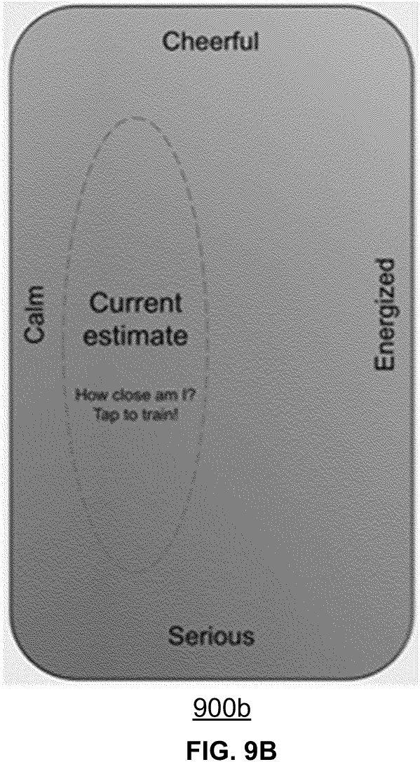

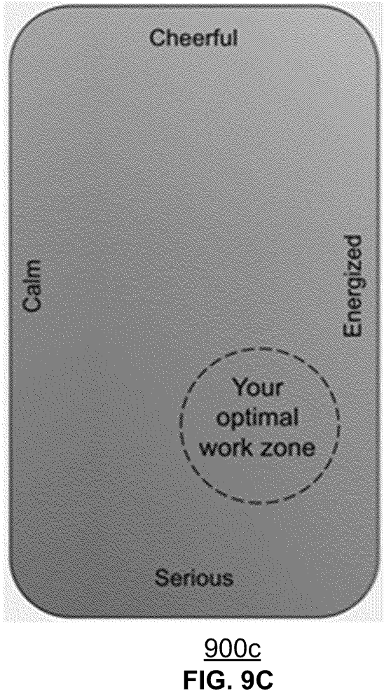

FIG. 9A depicts an example graphical user interface 900a that shows the axes of an example valence-arousal state space. As shown, the horizontal axis may indicate the arousal state (with energy level ranging from calm to energized). The vertical axis may indicate emotional state (ranging from serious to cheerful).

Although interface 900a (and several other examples) shows a multi-dimensional (i.e., two dimensions) mental state space where, for example, the first dimension is emotion and the second dimension is arousal (related to subjectively felt “energy”). Other two-dimensional state spaces may employ different dimensions. Likewise, some embodiments may employ more than two dimensions.

Some embodiments are configurable to enable a user to select one or both of (i) the number of dimensions (e.g., 1, 2, 3, 4, or more dimensions), and (ii) the mental state attribute corresponding to each dimension (e.g., emotion, energy, motivation, relaxation, and so on). In some embodiments, the number of dimensions and the mental state attribute corresponding to each dimension may be based on one or both of (i) an activity selected by the user (e.g., focus, meditation, relaxation, sleep, and so on), and (ii) attributes of the media items available to be played.

Similarly, while interface 900a (and several other examples) show a multi-dimensional mental state space, other embodiments may instead employ a multi-dimensional music state space. In embodiments that additionally or alternatively employ a multi-dimensional music state space, the dimensions correspond to musical aspects rather than mental states.

For example, an embodiment that employs a multi-dimensional music state space rather than a multi-dimensional mental state space may include a first musical attribute dimension (e.g., soft to loud) and a second musical attribute (e.g., simple to complex). In such embodiments, the position of an audio track within the multi-dimensional music state space corresponds to the audio track’s corresponding loudness and complexity.

If a user wants to alter an audio track over time, the user specifies a desired trajectory for the alteration. This desired music alteration trajectory is similar to the desired mental state trajectory described above. But rather than specifying a desired change from an initial mental state to a target mental state, the desired music alteration trajectory specifies a desired change from one or more initial musical attributes (e.g., soft and complex) to one or more target musical attributes (e.g., loud and simple). In operation, causing an audio track to follow the desired music alteration trajectory causes the audio track (e.g., which is initially soft and complex) to change, e.g., over some timeframe, which might be user specified (e.g., to being loud and simple).

Although the musical state space is described above with reference to two dimensions, some embodiments may employ musical state spaces having more than two dimensions. Similarly, some embodiments may use musical attribute axes that are different from the loud/soft and simple/complex axes described above. For example, other musical attribute axes may include fast/slow, harmonic/inharmonic, bright/dark, and so on.

As described herein, changing one or more musical attributes of an audio track may also change an expected trajectory through a multi-dimensional mental state space. However, implementing a desired music alteration trajectory through a musical state space is different than using a music track to implement a desired trajectory of a user’s mental state through a multi-dimensional mental state space because the former deals with changing musical attributes of an audio track whereas the latter deals with changing a user’s mental state.

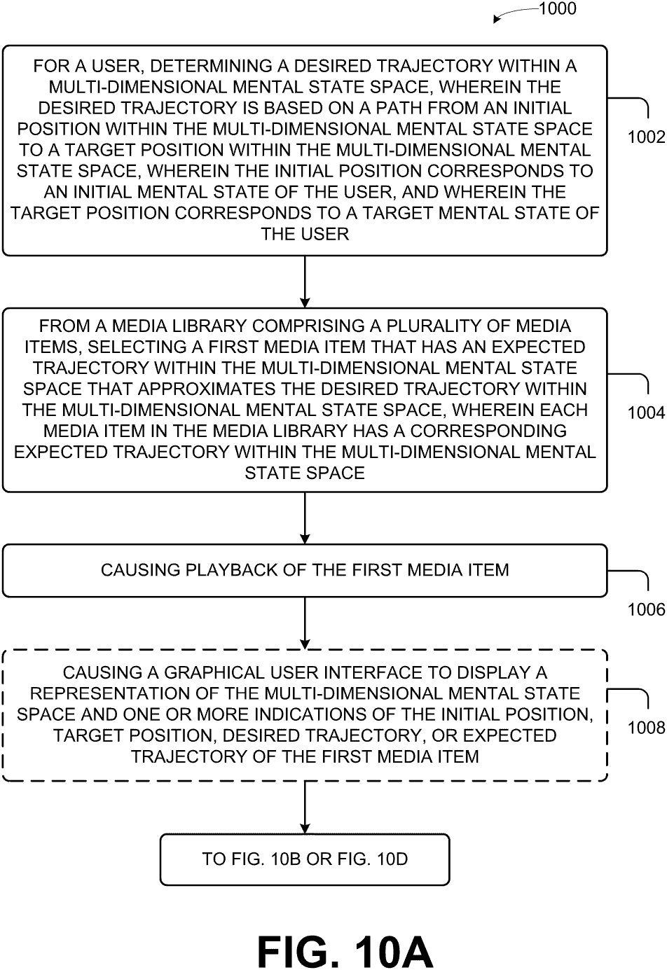

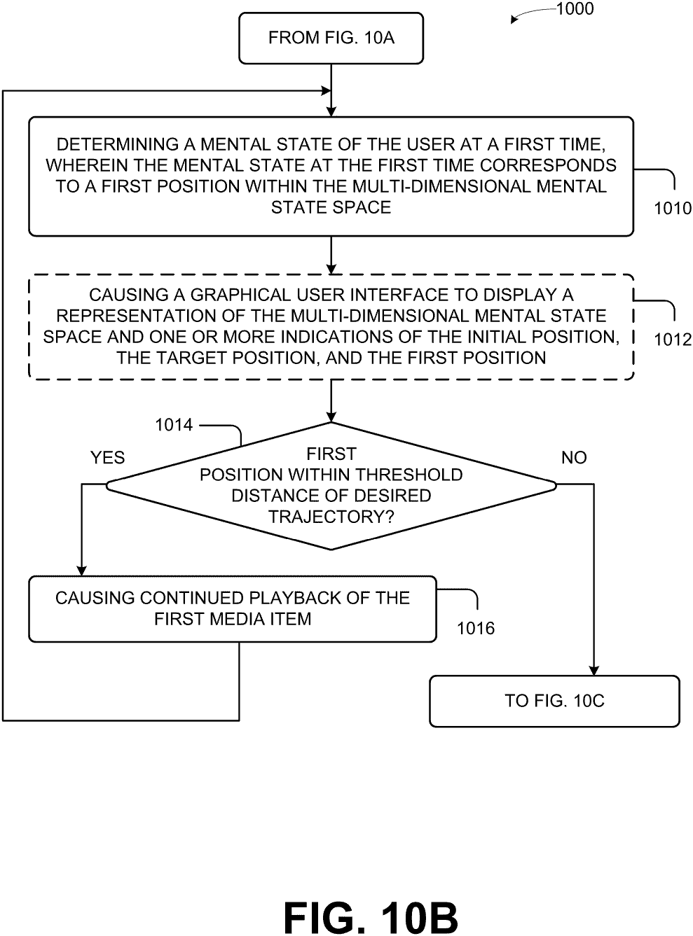

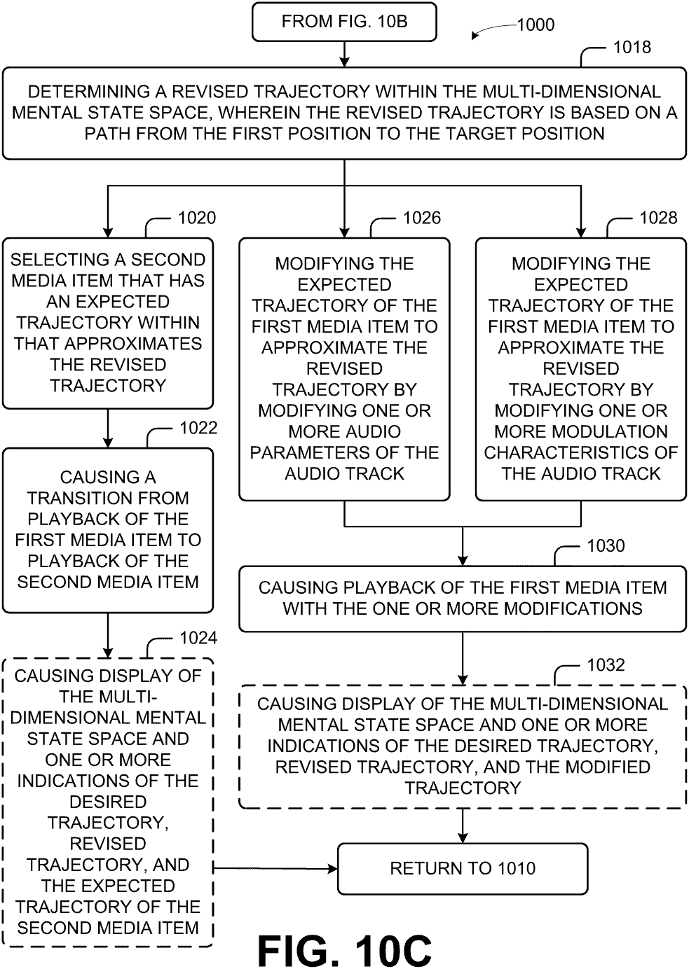

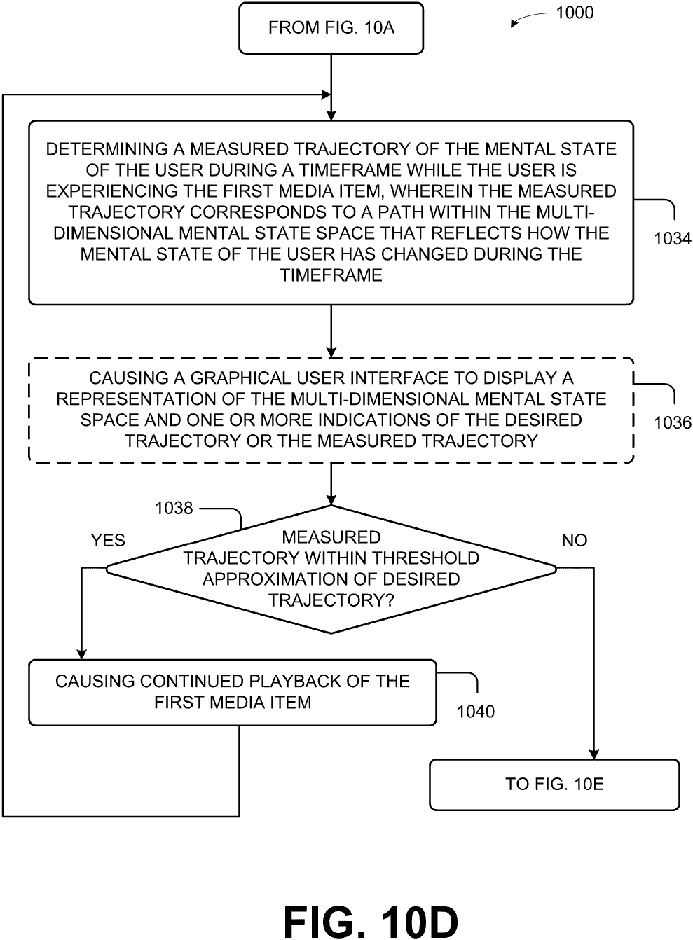

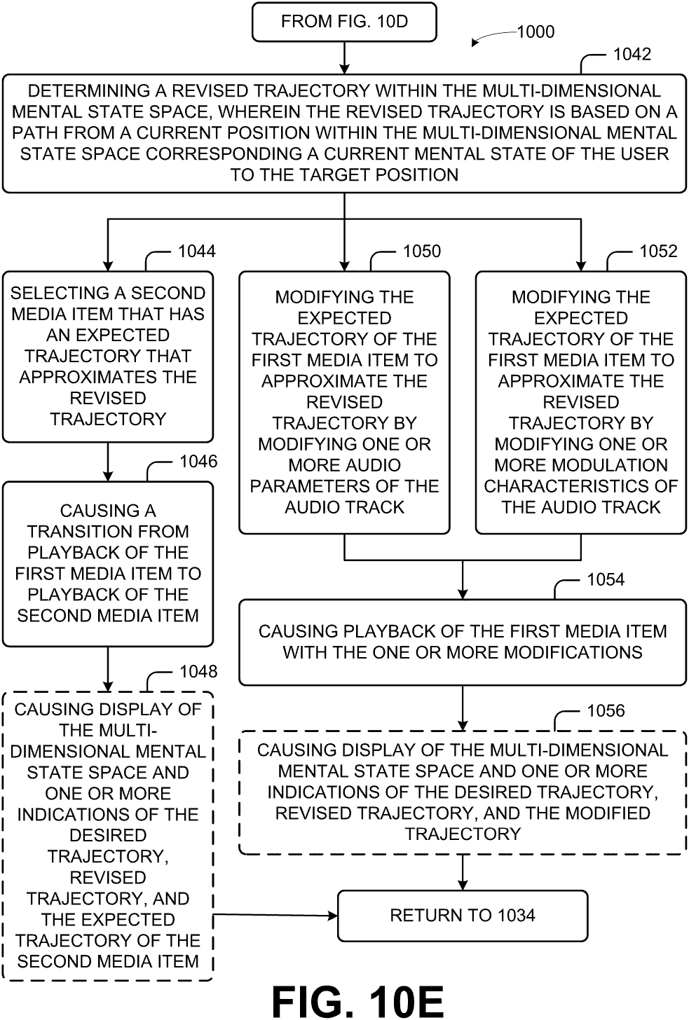

Nevertheless, some embodiments use desired music alteration trajectories through a musical state space in combination with implementing desired mental state trajectories of a user’s mental state through a multi-dimensional mental state space. For example, some embodiments may implement a desired music alteration trajectory through a musical state space to transition an audio track from a first position within the musical state space to a second position within the musical state space as part of using the modifications to the audio track to implement a revised mental state trajectory through the multi-dimensional mental state space for a user. Details of implementing a revised mental state trajectory are described in more detail herein with reference to FIGS. 10A-E.

FIG. 9B depicts another example graphical user interface 900b that shows a user a current estimate of his/her position (corresponding to the user’s current mental state) in the multi-dimensional mental state space. As shown, the current estimate may be a region within the multi-dimensional mental state space. As described herein, a region (like the region shown in FIG. 9B) within the multi-dimensional mental state space may include several points (like the points shown in FIGS. 7, and 8A-B). In some embodiments, the region may be in one dimension, e.g., the closed loop system may be able to predict the valence levels but not the arousal levels. In one example, a single dimension in which the system may operate is a ‘stress measurement’ which may be derived from sensor-data and/or user input. In the one-dimensional case, the user display on the interface may not be a plane but instead may be a line, meter, slider, or other one-dimensional indicator.

Based on the user inputs to the interface, the closed loop system may be trained and/or refined (depicted in FIG. 9B). For instance, the user input may be used to confirm the prediction based on sensor data. In another instance, a prediction along a dimension (e.g., valence state) may be displayed and the user may be prompted to confirm the valence state and further provide an input on the arousal state. The confirmation and the additional input for the arousal state may be used to train and/or refine the closed loop system. The user interface may also allow the users to enter their target states, and later indicate if and when the target states are reached.

Historical patterns may be tracked to identify and/or predict optimal mental states for users and the interface may be used for recommending the optimal states. For example, if a user indicates to the closed loop system the times when they feel they are in the optimal work state, the interface may show the state in the user interface, as shown in the example graphical user interface 900c depicted in FIG. 9C. Based on the identified and/or predicted optimal mental states for the user, the user may be pushed toward that state during a corresponding time. For instance, the closed loop system may push the user toward an optimal work zone during regular business hours.

In some embodiments, when the user selects his/her desired state (e.g., a desired target state), an interface may show the current state of the user. The interface may further show one or more trajectories, such as (i) a desired trajectory showing a path through the multi-dimensional mental state space from the user’s initial position (corresponding to the user’s initial mental state) to the user’s desired target position (corresponding to the user’s target mental state, (ii) an expected trajectory of an audio track showing how the audio track is expected to affect the user’s mental state, (iii) a revised trajectory showing a revised path through the mental state from the user’s current position (corresponding to the user’s current mental state while listening to an audio track) to the user’s desired target position, and/or (iv) a modified expected trajectory of an audio track showing how the audio track is expected to affect the user’s mental state after one or more attributes of the audio track (i.e., one or more audio parameters and/or modulation characteristics) have been modified.

As described herein a revised trajectory may be implemented by changing to different audio tracks, or manipulating audio within one track. Changing audio within a single track may be done by changing the rules governing music generation (if the music is being generated), or by signal processing techniques such as filtering and amplitude modulation. For example, the system may reduce the brightness (treble content) of the music over time as it discovers that this is effective at reducing stress for a particular person. The rules governing the search process (for effective music) can be guided by prior knowledge (research), and/or can be learned by the system.

Embodiments disclosed herein may further handle if the boundary conditions of the valence-arousal space are violated. For example, if a user falls asleep, the corresponding state may be beyond calm (as shown in the interfaces described above, left of the left boundary of the valence-arousal space). This boundary condition violation may be handled by special behavior (which may be based on the user preferences), such as playing audio to wake the user up or continuing with the current audio to keep the user asleep. Similarly, if a user gets over excited with the state to the right of the right boundary of the valence-arousal state, as detected by a spiked heart rate, the audio may be switched to relaxing music to move the user towards the normal valence-arousal boundaries. Additionally or alternatively, the user may be provided a message requesting him/her to take a moment for themselves when they can.

In addition to handling boundary condition violations, the system may provide other specific mental state transitions. For instance, when a user’s heart rate has been below 75 bpm for a period of time (e.g., three minutes) indicating a physiologically restful state, the audio may be modified/changed to a more energizing audio.

Embodiments disclosed herein may not just move the users from one valence-arousal location to a desired valence-arousal location. Embodiments disclosed herein may further allow the user to maintain a desired valence-arousal state once it is achieved. For example, based on collected sensor data, the closed loop system may subtly modify the audio being played to maintain the current (and desired) valence-arousal state of the user.

In some embodiments, the user’s state may not be continuously monitored, but measured at particular times. This may be requested by the user. For example, the user may select to test his/her stress level and, upon determining it is high, may then begin a relaxation program (e.g., with 5 minutes of music), after which their stress level is measured again. Some embodiments may not involve a ‘fully closed loop’ system in that the audio may not change in response to the sensor data in real time. For example, in one embodiment, a stress metric may be tracked during audio, and a history of audio and resulting stress is recorded. This history may be used to guide future choices (e.g., by selecting the best audio) or generate new audio (e.g., through modification).

In one embodiment, sensors detect when the user’s state is less sensitive to the stimulus (e.g., a decreasing heart rate due to music has “bottomed out”), and the system may change the stimulus in response. For example, it may be determined that physiological changes in response to relaxing music are “complete” after 2 minutes, but that then switching to silence, or some other music, can at that point drive physiological changes even farther (e.g., a yet greater reduction in heart rate). In one embodiment, users are reminded to listen to an audio (e.g., direct their attention to the music) at predetermined intervals or when sensor data indicate the user’s mental state has become less sensitive to the stimulus (e.g., following the onset of relaxing music, heart rate may drop but then level out and rise again; these effects may be influenced by attention or adaptation). These embodiments are just some examples of some use cases and should not be considered limiting.

### J. Example Methods for Audio Content Serving and Creation

FIGS. 10A-E show process flowcharts according to example embodiments of the present disclosure. The method 1000 may include one or more operations, functions, or actions as illustrated in one or more of blocks 1002-1056. Although the blocks are illustrated in sequential order, these blocks may also be performed in parallel, and/or in a different order than the order disclosed and described herein. Also, the various blocks may be combined into fewer blocks, divided into additional blocks, and/or removed based upon a desired implementation

FIG. 10A shows a first portion of a method 1000 for audio content serving and creation based on modulation characteristics and closed loop monitoring according to some example embodiments. Method 1000 can be performed by any of the computing devices and/or computing systems disclosed and/or described herein, individually or in combination with each other and/or other computing devices or systems. For example, method 1000 can be performed by any of the computing devices and/or systems shown and described with reference to FIGS. 5 and 6 or any other computing device and/or computing system that includes (i) one or more processors, and (ii) tangible, non-transitory, computer-readable media with program instructions stored thereon, where the program instructions, when executed by the one or more processors, cause the computing device and/or computing system to perform the functions of method 1000. Similarly, some embodiments include tangible, non-transitory, computer-readable media with program instructions for performing the functions of method 1000.

Method 1000 begins at block 1002, which includes, for a user, determining a desired trajectory within a multi-dimensional mental state space. In some embodiments, the desired trajectory is based on a path from an initial position within the multi-dimensional mental state space to a target position within the multi-dimensional mental state space. The initial position corresponds to an initial mental state of the user, and the target position corresponds to a target mental state of the user.

In some embodiments, a target region of the multi-dimensional mental state space corresponds to an activity selected from a set of activities comprising, for example: (i) focus, (ii) meditation, (iii) relaxation, and (iv) sleep. In some examples, the target region of the multi-dimensional mental state space can be indicated by what the user wants to do, a particular activity, a type of work, what time of day it is, and so on. Thus, in some examples, one or more target regions of the multi-dimensional mental state space are mapped to different activities or tasks. Mapping target activities and tasks to regions within the multi-dimensional mental state space enables disclosed embodiments to map a desired user activity (e.g., deep work) to a particular region within the multi-dimensional mental state space, e.g., a serious and energized mental state, corresponding to the “optimal work zone” shown in the bottom right region of the example user interface of FIG. 9C.

In some embodiments, determining the desired trajectory within the multi-dimensional mental state space at block 1002 includes receiving an indication of the desired trajectory from the user.

In some embodiments, determining the desired trajectory within the multi-dimensional mental state space at block 1002 includes: (i) determining the initial mental state of the user based on at least one of (a) an input from the user indicating the initial mental state or (b) sensor data relating to the initial mental state; (ii) determining the target mental state of the user based on at least one of (a) an input from the user indicating the target mental state or (b) sensor data relating to the target mental state; and (iii) determining the desired trajectory based on (a) the initial position within the multi-dimensional mental state space corresponding to the initial mental state of the user and (b) the target position within the multi-dimensional mental state space corresponding to the target mental state of the user.

In some embodiments, the desired trajectory can be determined via a single input (e.g., a user input or sensor input) that indicates both the initial mental state of the user and the target mental state of the user. For example, a sensor input from a GPS sensor (e.g., on the user’s smartphone) may indicate that the user has arrived at work, which may result in determining a desired trajectory towards the “optimal work zone” region in the bottom right hand corner of the user interface shown in FIG. 9C.

In another example, the desired trajectory can be determined via two inputs, e.g., GPS data from the user’s smartphone showing that the user just arrived at work, and heart rate data from the user’s smartwatch showing that the user’s hear rate is low. This combination of sensor inputs may result in determining a desired trajectory towards the “optimal work zone” region, but perhaps moving further to the right (toward “energized”) before moving downward (toward “serious”).

In some embodiments, the desired trajectory is a trajectory through a two-dimensional mental state space similar to the trajectories and multi-dimensional mental state spaces shown and described with reference to the examples shown in FIGS. 7, 8A-B, and 9A-C. However, in some embodiments, the desired trajectory is a trajectory through a 3, 4, or higher dimension mental state space.

As mentioned earlier, some embodiments enable a user to select one or both of (i) the number of dimensions (e.g., 1, 2, 3, 4, or more dimensions), and (ii) the mental state attribute corresponding to each dimension (e.g., emotion, energy, motivation, relaxation, and so on). In some embodiments, the number of dimensions and/or the mental state attribute corresponding to each dimension may be based on one or both of (i) an activity selected by the user (e.g., focus, meditation, relaxation, sleep, and so on), and (ii) attributes of the media items available to be played.

In some embodiments, a higher-dimension mental state space may be represented to a user in the form of a series of one-dimensional representations such as sliders or dials (as an example), where each slider or dial controls and/or displays a level within a different dimension, e.g., a separate dial/slider for emotion, energy, motivation, relaxation, and so on.

Next, method 1000 advances to block 1004, which includes selecting, from a media library comprising a plurality of media items, a first media item that has an expected trajectory within the multi-dimensional mental state space that approximates the desired trajectory within the multi-dimensional mental state space.

In operation, each media item in the media library has a corresponding expected trajectory within the multi-dimensional mental state space. The expected trajectory of a media item may be independent of the starting position in the space, or may be conditional on it. As an example of the latter case, a particular track may shift a user 10 units up and 10 units right if they start in the lower left hand corner of the space, but may shift them 8 units up and 5 units right if they start in the middle of the space.

In some embodiments, determining whether the expected trajectory of a media item approximates the desired trajectory includes comparing features of the expected trajectory with features of the desired trajectory, where an expected trajectory having features similar to the desired trajectory is deemed to approximate the desired trajectory. For example, if the desired trajectory is upward 5 units and to the left 3 units within the multi-dimensional mental state space, an expected trajectory (for a media item) that approximates the desired trajectory should also be upward about 5 units and to the left about 3 units.

It may be advantageous in some scenarios to combine several media items to create an expected trajectory that approximates the desired trajectory. To follow the above-described example, it may be advantageous in some instances to create an expected trajectory from two media items, e.g., combining (i) a first media item with a trajectory that is upward about 5 units and to the left about 1 unit and (ii) a second media item with a trajectory that is to the left about 2 units. In the way the combination of the trajectories of the first and second media items approximates the desired trajectory. In operation, 2, 3, 4, or more media items, each with its own expected trajectory, can be combined to create a set of two or more media items having a combined expected trajectory based on the expected trajectories of the individual media items within the set.

In some embodiments, a media item may already be playing (i.e., a currently playing media item) when the desired trajectory is determined at block 1002. In such a scenario, rather than selecting a first media that has an expected trajectory within the multi-dimensional mental state space, block 1004 may instead include modifying the currently playing media item to have an expected trajectory that approximates the desired trajectory.

Some embodiments of block 1004 may include selecting several media items that each have different expected trajectories explained above. For example, a first media item may have a first expected trajectory and a second media item may have a second expected trajectory, and in operation, the combination of the first trajectory and the second trajectory yields an expected trajectory for the combination that approximates the desired trajectory.

In some embodiments, rather than selecting a first media that has an expected trajectory within the multi-dimensional mental state space, block 1004 may instead include generating a first media item that has an expected trajectory that approximates the desired trajectory. In this manner, generating a first media item may include selecting one or more media items and/or modifying the one or more selected media items to achieve an expected trajectory by (i) modifying one or more audio parameters of the selected media item(s) and/or (ii) modifying one or more modulation characteristics of the selected media(s).

Next, method 1000 advances to block 1006, which includes causing playback of the first media item. In some embodiments, causing playback of the first media item may include playing the first item via the same computing device that is performing one or more functions of method 1000, e.g., a smartphone, tablet, laptop, or other computing device. In some embodiments, causing playback of the first media item includes causing a device separate from the computing device performing one or more functions of method 1000 to play the first media item, e.g., a separate speaker system, headphones, another computing device comprising speakers, or similar. Blocks 1002, 1004, and 1006 may or may not happen in quick temporal succession, and may or may not be triggered by completion of the previous block. For example, the system may determine a desired trajectory (block 1002) at a point in time when it does not have access to a media library; in this case, selection of media (block 1004) and playback (block 1006) may then occur at a later time.

Some embodiments of method 1000 optionally include block 1008, which includes causing a graphical user interface to display a representation of the multi-dimensional mental state space and one or more indications of the initial position, target position, desired trajectory, and/or expected trajectory of the first media item.

For example, some embodiments of block 1008 include causing a graphical user interface to display a representation of the multi-dimensional mental state space and one or more of (i) an indication of the initial position within the multi-dimensional mental state space corresponding to the initial mental state of the user or (ii) an indication of the target position within the multi-dimensional mental state space corresponding to the target mental state of the user.

Some examples additionally include causing the graphical user interface to display the user’s stored history of trajectories through the multi-dimensional mental state space. Enabling the user to view a history of stored trajectories along with media items that the user experienced during those past trajectories can help the user better understand which media items have worked better for certain trajectories. Some embodiments additionally include using such historical trajectories (and media items associated therewith) to improve the selection and/or modification of media items for implementing desired trajectories in the future.

Some embodiments of block 1008 include causing a graphical user interface to display a representation of the multi-dimensional mental state space and one or more of (i) an indication of the desired trajectory or (ii) an indication of the expected trajectory corresponding to the first media item.

Next, some embodiments of method 1000 advance to FIGS. 10B-C, whereas other embodiments alternatively advance to FIGS. 10D-E. The features and functionality of the embodiments shown in FIGS. 10B-C are described next, followed by the features and functionality of the embodiments shown in FIGS. 10D-E.

Embodiments of method 1000 shown in FIGS. 10B-C include block 1010, which includes determining a mental state of the user at a first time, wherein the mental state at the first time corresponds to a first position within the multi-dimensional mental state space. In some embodiments of block 1010, determining the mental state of the user at the first time is based on at least one of (i) an input from the user indicating the mental state of the user at the first time, or (ii) sensor data relating to the mental state of the user at the first time. Although the description of method 1000 uses the term “first position,” the process executed in method 1000 is iterative as shown in FIG. 10B. As such, use of the term “first position” in block 1010 (and other blocks) is intended to include each position determined at block 1010 in each successive iteration of the steps in method 1000.

After block 1010, method 1000 advances to optional method block 1012, which includes causing a graphical user interface to display a representation of the multi-dimensional mental state space and one or more indications of the initial position, the target position, and/or the first position.

For example, some embodiments of block 1012 include causing a graphical user interface to display a representation of the multi-dimensional mental state space and one or more of (i) an indication of the initial position within the multi-dimensional mental state space corresponding to the initial mental state of the user, (ii) an indication of the target position within the multi-dimensional mental state space corresponding to the target mental state of the user, (iii) an indication of the first position within the multi-dimensional mental state space corresponding to the mental state of the user at the first time.

After block 1012, method 1000 advances to block 1014, which includes determining whether the first position (from block 1010) is within a threshold distance of the desired trajectory (from block 1002).

At block 1014, when the first position is within the threshold distance of the desired trajectory within the multi-dimensional mental state space, then method 1000 advances to block 1016, which includes causing continued playback of the first media item. In operation, determining whether the first position is within the threshold distance of the desired trajectory may be performed by any suitable method of determining a distance between a point in space and a line because the first position is represented as a point in the multi-dimensional mental state space and the desired trajectory is represented as a line (or path) through the multi-dimensional mental state space.

When the first position is within the threshold distance of the desired trajectory, then playback of the first media item is likely causing the mental state of the user to progress along the desired trajectory from the initial position (corresponding to the initial mental state of the user) to the target position (corresponding to the target mental state). Accordingly, continued playback of the first media item should continue to cause the mental state of the user to progress along the desired trajectory toward the target mental state.

After causing the continued playback of the first media item at block 1016, method 1000 returns to block 1010, which includes determining the mental state of the user again and continuing execution of method 1000.

If at block 1014, the first position is not within the threshold distance of the desired trajectory within the multi-dimensional mental state space, then the method advances to block 1018 (FIG. 10C).

Block 1018 includes determining a revised trajectory within the multi-dimensional mental state space, wherein the revised trajectory is based on a path from the first position to the target position.

Some embodiments of method 1000 include one or more of several alternatives after determining a revised trajectory within the multi-dimensional mental state space at block 1018. For embodiments where the media item is an audio track, the several alternatives include (i) selecting a new audio track (block 1020), (ii) modifying audio parameters of an audio track (block 1026), and (iii) modifying modulation characteristics (block 1028). These alternatives are described further herein.

In some embodiments, after determining a revised trajectory within the multi-dimensional mental state space at block 1018, method 1000 advances to block 1020, which includes selecting a second media item that has an expected trajectory within the multi-dimensional mental state space that approximates the revised trajectory (from block 1018).

After selecting a second media item that has an expected trajectory within the multi-dimensional mental state space that approximates the revised trajectory at block 1020, method 1000 advances to block 1022, which includes causing a transition from playback of the first media item to playback of the second media item. This transition may include a cross-fade from one media item to the next, a stopping of one before the other begins (with silence between), a constructed transition designed to minimize jarring discontinuities, or other methods of transitioning between media items.

Some embodiments optionally include method block 1024, which includes causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of the expected trajectory corresponding to the second media item.

After 1022 (or optionally block 1024), method 1000 returns to block 1010, which includes determining the mental state of the user again and continuing execution of method 1000.

In some embodiments, after determining a revised trajectory within the multi-dimensional mental state space at block 1018, method 1000 advances to block 1026, which includes modifying the expected trajectory of the first media item to approximate the revised trajectory by modifying one or more audio parameters of the audio track.

In some embodiments, the first media item includes an audio track. In such embodiments, the expected trajectory corresponding to the first media item is based on (i) one or more audio parameters of the audio track and/or (ii) one or more modulation characteristics of the audio track.

In some embodiments, modifying the expected trajectory of the first media item to approximate the revised trajectory by modifying one or more audio parameters of the audio track at block 1026 includes modifying one or more audio parameters or elements of the audio track resulting in the modification of: (i) a tempo of the audio track; (ii) a RMS (root mean square energy in signal) of the audio track; (iii) a loudness of the audio track; (iv) an event density of the audio track; (iv) a spectral brightness of the audio track; (v) a temporal envelope of the audio track or elements thereof; (vi) spectral envelope structure (measured as a cepstrum of the audio track); (vii) dominant pitches (measured via the chromagram); (viii) change over time (measured as flux of the audio track or other methods); (ix) regularity or self-similarity over time (measured via an autocorrelation of the audio track or other methods); (x) how acoustic energy within the audio track is distributed over spectral modulation rates (measured as a spectral modulation spectrum of the audio track); (xi) an attack and decay of the audio track that changes a rise and fall time of audio events within the audio track; (xii) textural aspects of audio such as roughness; (xiii) a degree of harmonicity/inharmonicity of the audio track; or (xiv) a sparseness of the audio track.

For example, the tempo of the audio track is the speed or pace of a piece of music, often given in beats per minute. Faster music tends to be energizing and associated with positive emotional valence (all else being equal). Thus, increasing the tempo of an audio track tends to alter the expected trajectory of the audio track toward higher energy within the multi-dimensional mental state space, e.g., towards to the right in the user interface examples shown in FIGS. 7, 8A-B, and 9A-C. Conversely, decreasing the tempo of the audio track tends to alter the expected trajectory of the audio track toward lower energy (i.e., calmer) within the multi-dimensional mental state space, e.g., towards the left in the user interface examples shown in FIGS. 7, 8A-B, and 9A-C.

In a further example, RMS of the audio track is a measure of energy in the audio signal. A track with a higher RMS will typically be louder, all else being equal. As a result, the RMS of the audio track is related to the loudness of the audio track. Loudness is a perceptual property of sound related to energy in the signal. Louder music is generally more energizing / stimulating, all else being equal. However, louder music can sometimes be distracting, which is an effect that depends on personality and perhaps other factors. Nevertheless, increasing the RMS and/or loudness of an audio track tends to alter the expected trajectory of the audio track toward higher energy within the multi-dimensional mental state space, e.g., towards to the right in the user interface examples shown in FIGS. 7, 8A-B, and 9A-C. Conversely, decreasing the RMS and/or loudness of the audio track tends to alter the expected trajectory of the audio track toward lower energy (i.e., calmer) within the multi-dimensional mental state space, e.g., towards the left in the user interface examples shown in FIGS. 7, 8A-B, and 9A-C.

In another example, the event density of an audio track describes how “busy” the music is, in terms of notes, instrumentation, or other sound events. Music with high event density is more stimulating but can sometimes be more distracting. Flux is similar to event density in that, like event density, flux is also a measure of how the music in the audio track changes over time. Increasing the event density and/or flux of an audio track tends to alter the expected trajectory of the audio track toward higher energy within the multi-dimensional mental state space, e.g., towards to the right in the user interface examples shown in FIGS. 7, 8A-B, and 9A-C. Conversely, decreasing the event density and/or flux of the audio track tends to alter the expected trajectory of the audio track toward lower energy (i.e., calmer) within the multi-dimensional mental state space, e.g., towards the left in the user interface examples shown in FIGS. 7, 8A-B, and 9A-C.

In a further example, the brightness of an audio track corresponds to the treble to bass balance of the sound. Bright, shiny sounds are typically calming and associated with positive emotional valence, but they may also be more distracting to some users. Increasing the spectral brightness of an audio track tends to alter the expected trajectory of the audio track toward lower energy (i.e., calmer) within the multi-dimensional mental state space, e.g., towards to the left in the user interface examples shown in FIGS. 7, 8A-B, and 9A-C.

In another example, the temporal envelope of an audio track describes how loudness changes over time on a very short timescale. For example the attack, sustain, decay, and release of a note (‘ASDR’ envelope) is a typical way of describing temporal envelope in music production. A temporal envelope that is relatively flat will have less change in loudness over time, and it may be calmer or less intrusive/distracting than a fluctuating envelope. Thus, modifying an audio track to have a more consistent temporal envelope tends to alter the expected trajectory of the audio track toward lower energy (i.e., calmer) within the multi-dimensional mental state space, e.g., towards to the left in the user interface examples shown in FIGS. 7, 8A-B, and 9A-C.

In yet another example, the chromagram of an audio track shows which pitches tend to dominate in the audio track. Audio with a single or only a few dominant pitches will be simpler, easier to parse, and less musically complex. The chromagram may show if the dominant pitches are consonant or dissonant, which can be associated with emotional valence (positive and negative, respectively). Thus, modifying an audio track so that the dominant pitches are more consonant tends to alter the expected trajectory of the audio track toward higher valence within the multi-dimensional mental state space, e.g., towards to the top in the user interface examples shown in FIGS. 7, 8A-B, and 9A-C. Likewise, modifying an audio track so that the dominant pitches are more dissonant may tend to alter the expected trajectory of the audio track toward lower valence within the multi-dimensional mental state space, e.g., towards to the bottom in the user interface examples shown in FIGS. 7, 8A-B, and 9A-C.

In yet another example, autocorrelation of the audio track reflects self-similarity of the audio track. A self-similar audio track is more predictable, easier to ignore/work to, and typically more calming (less jarring), than music that changes often. Thus, modifying an audio track to increase its self-similarity may tend to alter the expected trajectory of the audio track to the bottom (more serious) and left (more calming) in the user interface examples shown in FIGS. 7, 8A-B, and 9A-C. Likewise, modifying an audio track to reduce its self-similarity may tend to alter the expected trajectory of the audio track to the top (more cheerful / less serious) and right (more energized / less calming) in the user interface examples shown in FIGS. 7, 8A-B, and 9A-C.

Thus, by modifying one or more audio parameters of an audio track (at block 1026), some embodiments can modify the expected trajectory of the audio track.

After modifying the expected trajectory of the first media item to approximate the revised trajectory by modifying one or more audio parameters of the audio track at block 1026, method 1000 advances to block 1030, which includes causing playback of the first media item with the one or more modifications, e.g., causing playback of the first media item with the one or more modified audio parameters.

Some embodiments optionally include method block 1032, which includes causing display of the multi-dimensional mental state space and one or more indications of the desired trajectory, revised trajectory, and the modified trajectory. For example, some embodiments of method block 1032 include causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more audio parameters of the audio track.

After block 1030 (or optional block 1032), method 1000 returns to block 1010, which includes determining the mental state of the user again and continuing execution of method 1000.

In some embodiments, after determining a revised trajectory within the multi-dimensional mental state space at block 1018, method 1000 advances to block 1028, which includes modifying the expected trajectory of the first media item to approximate the revised trajectory by modifying one or more modulation characteristics of the audio track.

In some embodiments, modifying the expected trajectory of the first media item to approximate the revised trajectory by modifying one or more modulation characteristics of the audio track at block 1028 includes modifying one or more modulation characteristics of the audio track or elements thereof (e.g. individual instruments of frequency regions) by modifying one or more of: (i) a modulation depth at a single modulate rate; (ii) a modulation rate; (iii) a plurality of modulation depths at a corresponding plurality of modulation rates; (iv) a modulation phase; or (v) a modulation waveform shape.

For example, the modulation depth of the audio track is the degree of amplitude fluctuation in the modulation added to the audio track. A greater modulation depth can help some users (e.g., users with ADHD) increase their focus. Thus, increasing the modulation depth of an audio track tends to alter the expected trajectory of the audio track within the multi-dimensional mental state space in a way that helps some users focus, e.g., move from their current mental state to the left (more calming) and bottom (more serious) in the user interface examples shown in FIGS. 7, 8A-B, and 9A-C.

In another example, the modulation rate of the audio track describes the rate of the modulation added to the audio track. A higher modulation rate tends to be more energizing, whereas a lower modulation tends to be more calming. Thus, increasing the modulation rate of the modulation added to the audio track tends to alter the expected trajectory of the audio track toward higher energy within the multi-dimensional mental state space, e.g., to the right in the user interface examples shown in FIGS. 7, 8A-B, and 9A-C. Conversely, decreasing the modulation rate of the modulation added to the audio track tends to alter the expected trajectory of the audio track toward lower energy (i.e., calmer) within the multi-dimensional mental state space, e.g., to the left in the user interface examples shown in FIGS. 7, 8A-B, and 9A-C.

Thus, by alternatively modifying one or more modulation characteristics of an audio track (at block 1028), some embodiments can modify the expected trajectory of the audio track.

Blocks 1026 and 1028 implement different features, i.e., modifying the expected trajectory of an audio track via modifying audio parameters (block 1026) versus modifying the expected trajectory of an audio track via modifying modulation characteristics (block 1028). However, modifying audio parameters and modifying modulation characteristics are not mutually exclusive functions. Thus, some embodiments may include modifying audio parameters and modulation characteristics for a particular audio track to modify the audio track’s expected trajectory.

After modifying the expected trajectory of the first media item to approximate the revised trajectory by modifying one or more modulation characteristics of the audio track at block 1028, method 1000 advances to block 1030 which includes causing playback of the first media item with the one or more modifications, e.g., causing playback of the first media item with the one or more modified modulation characteristics. The playback of the modified media may occur by transitioning from unmodified to modified media (via cross-fading or other methods), or may occur in the course of continuous playback and real-time processing of the media i.e., that the media is modified ‘on the fly’ and there are not two separate media items.

As mentioned above, some embodiments optionally include method block 1032, which includes causing display of the multi-dimensional mental state space and one or more indications of the desired trajectory, revised trajectory, and the modified trajectory. For example, some embodiments of block 1032 include causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more modulation characteristics of the audio track.

After block 1030 (or optional block 1032), method 1000 returns to block 1010, which includes determining the mental state of the user again and continuing execution of method 1000.

By monitoring and determining the user’s mental state at block 1010, and then determining a revised trajectory at block 1018 when the user’s mental state has veered from the desired trajectory (determined at block 1002), method 1000 is able to keep guiding the user’s mental state toward the target mental state. Additionally, after the user has achieved the target mental state, embodiments of method 1000 include monitoring the user’s mental state at block 1010, and then determining (at block 1014) a revised trajectory when the user’s mental state has strayed from the target mental state (that the user previously achieved). This revised trajectory determined at block 1014 can then be used to guide the user’s mental state back to the target state.

As mentioned earlier, and shown in FIG. 10A, some embodiments of method 1000 advance to FIGS. 10B-C, whereas other embodiments alternatively advance to FIGS. 10D-E. The features and functionality of the embodiments shown in FIGS. 10D-E are described next.

Embodiments of method 1000 shown in FIGS. 10D-E include block 1034, which includes determining a measured trajectory of the mental state of the user during a timeframe while the user is experiencing the first media item. The measured trajectory corresponds to a path within the multi-dimensional mental state space that reflects how the mental state of the user has changed during the timeframe. In some embodiments of block 1034, determining the measured trajectory mental state of the user is based on a series of two or more (i) inputs from the user indicating a mental state of the user during the timeframe or (ii) sensor measurements during the timeframe.

Some embodiments optionally include block 1036, which includes causing a graphical user interface to display a representation of the multi-dimensional mental state space and one or more indications of the desired trajectory or the measured trajectory.

After determining a measured trajectory of the mental state of the user during a timeframe while the user is experiencing the first media item at block 1034 (and optionally displaying indications of the desired and/or measured trajectories at block 1036), method 1000 advances to block 1038, which includes determining whether the measured trajectory (from block 1034) is within a threshold approximation of at least some portion of the desired trajectory (from block 1002).

At block 1038, if the measured trajectory is within a threshold approximation of at least a portion of the expected trajectory within the multi-dimensional mental state space, then method 1000 advances to block 1040, which includes causing continued playback of the first media item.

After block 1040, method 1000 returns to block 1034, which includes determining the measured trajectory of the mental state of the user again and continuing execution of method 1000.

Determining whether the measured trajectory is within the threshold approximation of the expected trajectory at block 1038 is more sophisticated than determining whether the first position is within a threshold distance of the desired trajectory at block 1014 because the measured trajectory includes the current mental state of the user (and the current position within the multi-dimensional mental state space corresponding to the user’s current mental state) as well as the change of the user’s mental state over time (including the historical positions within the multi-dimensional mental state space corresponding to the user’s historical mental state). In this manner, determining the measured trajectory reveals how the user’s mental state is changing over time, including whether the user’s mental state is progressing toward the target state or perhaps reverting backwards toward the initial state (even if the user’s current state may still be within the threshold distance of the desired trajectory).

Because both the user’s current mental state and the user’s historical mental state are useful in assessing whether playback of the first media item is successfully causing the user’s mental state to progress from the initial state to the target state, some embodiments include making a determination about whether to continue playback of the first media item based on both (i) the user’s current mental state and (ii) the user’s historical mental state (as reflected in the measured trajectory).

When the measured trajectory at block 1038 is within the threshold distance of the desired trajectory, then playback of the first media item is likely causing the mental state of the user to progress along the desired trajectory from the initial position (corresponding to the initial mental state of the user) to the target position (corresponding to the target mental state). Accordingly, continued playback of the first media item should continue to cause the mental state of the user to progress along the desired trajectory toward the target mental state.

But if at block 1038, the measured trajectory is not within the threshold approximation of the expected trajectory, then method 1000 advances to block 1042 (FIG. 10E), which includes determining a revised trajectory within the multi-dimensional mental state space. In some embodiments, the revised trajectory is based on a path from (i) a current position within the multi-dimensional mental state space corresponding to a current mental state of the user to (ii) the target position within the multi-dimensional mental state space. In some embodiments, the current mental state of the user (and thus, the corresponding current position within the multi-dimensional mental state space) is based on at least one of (a) an input from the user indicating the current mental state, or (b) data from one or more sensors.

By monitoring and determining the user’s mental state trajectory at block 1034, and then determining a revised trajectory at block 1042 when the user’s mental state trajectory has veered from the desired trajectory (determined at block 1002), method 1000 is able to keep guiding the user’s mental state toward the target mental state. Additionally, after the user has achieved the target mental state, embodiments of method 1000 include monitoring the user’s mental state at block 1034, and then determining (at block 1042) a revised trajectory when the user’s mental state has strayed from the target mental state (that the user previously achieved). This revised trajectory determined at block 1042 can then be used to guide the user’s mental state back to the target state.

Some embodiments of method 1000 include one or more of several alternatives after determining a revised trajectory within the multi-dimensional mental state space at block 1042. For embodiments where the media item is an audio track, the several alternatives include (i) selecting a new audio track (block 1044), (ii) modifying audio parameters of an audio track (block 1050), and (iii) modifying modulation characteristics (block 1052). These alternatives are described further herein.

In some embodiments, after determining a revised trajectory within the multi-dimensional mental state space at block 1042, method 1000 advances to block 1044, which includes selecting a second media item that has an expected trajectory within the multi-dimensional mental state space that approximates the revised trajectory within the multi-dimensional mental state space.

After selecting a second media item that has an expected trajectory within the multi-dimensional mental state space that approximates the revised trajectory within the multi-dimensional mental state space at block 1044, method 1000 advances to block 1046, which includes causing a transition from playback of the first media item to playback of the second media item.

Some embodiments optionally include method block 1048, which includes causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of the expected trajectory corresponding to the second media item.

After block 1046 (or optional block 1048), method 1000 returns to block 1034, which includes determining the measured trajectory of the mental state of the user again and continuing execution of method 1000.

As mentioned earlier, in some embodiments, the first media item includes an audio track. In such embodiments, the expected trajectory corresponding to the first media item is based on (i) one or more audio parameters of the audio track and (ii) one or more modulation characteristics of the audio track.

In some embodiments, after determining a revised trajectory within the multi-dimensional mental state space at block 1042, method 1000 advances to block 1050, which includes modifying the expected trajectory of the first media item to approximate the revised trajectory by modifying one or more audio parameters of the audio track.

In some embodiments, modifying the expected trajectory of the first media item to approximate the revised trajectory by modifying one or more audio parameters of the audio track at block 1050 includes modifying one or more audio parameters or elements of the audio track resulting in the modification of: (i) a tempo of the audio track; (ii) a RMS (root mean square energy in signal) of the audio track; (iii) a loudness of the audio track; (iv) an event density of the audio track; (iv) a spectral brightness of the audio track; (v) a temporal envelope of the audio track or elements thereof; (vi) spectral envelope structure (measured as a cepstrum of the audio track); (vii) dominant pitches (measured via the chromagram); (viii) change over time (measured as flux of the audio track or other methods); (ix) regularity or self-similarity over time (measured via an autocorrelation of the audio track or other methods); (x) how acoustic energy within the audio track is distributed over spectral modulation rates (measured as a spectral modulation spectrum of the audio track); (xi) an attack and decay of the audio track that changes a rise and fall time of audio events within the audio track; (xii) textural aspects of audio such as roughness; (xiii) a degree of harmonicity/inharmonicity of the audio track; or (xiv) a sparseness of the audio track. Modifying one or more audio parameter of the first media item at block 1050 is similar to or the same as block 1026.

Blocks 1050 and 1052 implement different features, i.e., modifying the expected trajectory of an audio track via modifying audio parameters (block 1050) and modifying the expected trajectory of an audio track via modifying modulation characteristics (block 1052). However, modifying audio parameters and modifying modulation characteristics are not mutually exclusive functions. Thus, some embodiments may include modifying audio parameters and modulation characteristics for a particular audio track to modify the audio track’s expected trajectory.

After modifying the expected trajectory of the first media item to approximate the revised trajectory by modifying one or more audio parameters of the audio track at block 1050, method 1000 advances to block 1054, which includes causing playback of the first media item with the one or more modifications, e.g., causing playback of the first media item with the one or more modified audio parameters.

Some embodiments optionally include method block 1056, which includes causing display of the multi-dimensional mental state space and one or more indications of the desired trajectory, revised trajectory, and the modified trajectory. For example, some embodiments of method block 1056 include causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more audio parameters of the audio track.

After block 1054 (or optional block 1056), method 1000 returns to block 1034, which includes determining the measured trajectory of the mental state of the user again and continuing execution of method 1000.

In some embodiments, after determining a revised trajectory within the multi-dimensional mental state space at block 1042, method 1000 advances to block 1052, which includes modifying the expected trajectory of the first media item to approximate the revised trajectory by modifying one or more modulation characteristics of the audio track.

In some embodiments, modifying the expected trajectory of the first media item to approximate the revised trajectory by modifying one or more modulation characteristics of the audio track at block 1052 includes modifying one or more of: (i) a modulation depth at a single modulate rate; (ii) a modulation rate; (iii) a plurality of modulation depths at a corresponding plurality of modulation rates; (iv) a modulation phase; or (v) a modulation waveform shape. Modifying one or more modulation characteristics of the first media item at block 1052 is similar to or the same as block 1028.

After modifying the expected trajectory of the first media item to approximate the revised trajectory by modifying one or more modulation characteristics of the audio track at block 1052, method 1000 advances to block 1054 which includes causing playback of the first media item with the one or more modifications, e.g., causing playback of the first media item with the one or more modified modulation characteristics.

As mentioned above, some embodiments optionally include method block 1054, which includes causing display of the multi-dimensional mental state space and one or more indications of the desired trajectory, revised trajectory, and the modified trajectory. For example, some embodiments of block 1056 include causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more modulation characteristics of the audio track.

After block 1054 (or optional block 1056), method 1000 returns to block 1034, which includes determining the measured trajectory of the mental state of the user again and continuing execution of method 1000.

The valence-arousal space is described above just as one example of a mental state space and therefore should not be considered limiting. The disclosed embodiments are applicable to other state spaces. An example of other types of state spaces may include heart rate and heart rate variability space. Therefore, any dimension which may be estimable using the sensor data and which may be influenced by audio should be considered within the scope of this disclosure. Furthermore, the embodiments are not limited to audio, which is just provided as an example. Any stimulus for which the valence-arousal (or any other type of state) effects may be predicted should be considered within the scope of this disclosure. For example, a video library may be tagged with how the different videos may affect valence and arousal, and the videos or portions therefore (e.g., short videos) may be used to move the mental states of people around in a state space (e.g., valence-arousal space).

Furthermore, the dimensionality (i.e., two-dimensions) of the valence-arousal state or other mental state spaces is also just for illustration. The disclosed embodiments may be applicable to one-dimensional state space (e.g., as described above, only for one of valence or arousal). Another example of one-dimensional space may include stress level, as also described above. Furthermore, the embodiments may be applied to higher dimensional state spaces. For example, the embodiments may be applied to a user’s location and movement in a three-dimensional state space of valence, arousal, and fatigue. Therefore, state space with any order of dimensionality that may be estimated by the sensors and influenced by audio/video (and/or any other type of multimedia) should be considered within the scope of this disclosure.

Additional examples of the presently described method and device embodiments are suggested according to the structures and techniques described herein. Other non-limiting examples may be configured to operate separately or can be combined in any permutation or combination with any one or more of the other examples provided above or throughout the present disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant’s intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A method performed by a computing system, wherein the method comprises:
for a user, determining a desired trajectory within a multi-dimensional mental state space, wherein the desired trajectory is based on a path from an initial position within the multi-dimensional mental state space to a target position within the multi-dimensional mental state space, wherein the initial position corresponds to an initial mental state of the user, and wherein the target position corresponds to a target mental state of the user;
from a media library comprising a plurality of media items, selecting a first media item that has an expected trajectory within the multi-dimensional mental state space that approximates the desired trajectory within the multi-dimensional mental state space, wherein each media item in the media library has a corresponding expected trajectory within the multi-dimensional mental state space; and
causing playback of the first media item.

2. The method of claim 1, wherein determining the desired trajectory within the multi-dimensional mental state space comprises:
receiving an indication of the desired trajectory from the user.

3. The method of claim 1, wherein determining the desired trajectory within the multi-dimensional mental state space comprises:
determining the initial mental state of the user based on at least one of (i) an input from the user indicating the initial mental state or (ii) sensor data relating to the initial mental state;
determining the target mental state of the user based on at least one of (i) an input from the user indicating the target mental state or (ii) sensor data relating to the target mental state; and
determining the desired trajectory based on (i) the initial position within the multi-dimensional mental state space corresponding to the initial mental state of the user and (ii) the target position within the multi-dimensional mental state space corresponding to the target mental state of the user.

4. The method of claim 1, further comprising:
causing a graphical user interface to display a representation of the multi-dimensional mental state space and one or more of: (i) an indication of the initial position within the multi-dimensional mental state space corresponding to the initial mental state of the user, (ii) an indication of the target position within the multi-dimensional mental state space corresponding to the target mental state of the user; (iii) an indication of the desired trajectory, or (iv) an indication of the expected trajectory corresponding to the first media item.

5. The method of claim 1, further comprising, while the first media item is playing:
determining a mental state of the user at a first time, wherein the mental state at the first time corresponds to a first position within the multi-dimensional mental state space, and wherein determining the mental state of the user at the first time is based on at least one of (i) an input from the user indicating the mental state of the user at the first time, or (ii) sensor data relating to the mental state of the user at the first time.

6. The method of claim 5, further comprising:
causing a graphical user interface to display a representation of the multi-dimensional mental state space and one or more of (i) an indication of the initial position within the multi-dimensional mental state space corresponding to the initial mental state of the user, (ii) an indication of the target position within the multi-dimensional mental state space corresponding to the target mental state of the user, (iii) an indication of the first position within the multi-dimensional mental state space corresponding to the mental state of the user at the first time.

7. The method of claim 5, further comprising:
when the first position is within a threshold distance of the desired trajectory within the multi-dimensional mental state space, causing continued playback of the first media item; and
when the first position is not within the threshold distance of the desired trajectory within the multi-dimensional mental state space, (i) determining a revised trajectory within the multi-dimensional mental state space, wherein the revised trajectory is based on a path from the first position to the target position, (ii) selecting a second media item that has an expected trajectory within the multi-dimensional mental state space that approximates the revised trajectory, and (iii) causing a transition from playback of the first media item to playback of the second media item.

8. The method of claim 7, further comprising:
causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of the expected trajectory corresponding to the second media item.

9. The method of claim 5, wherein the first media item comprises an audio track, wherein the expected trajectory corresponding to the first media item is based on (i) one or more audio parameters of the audio track and (ii) one or more modulation characteristics of the audio track, and wherein the method further comprises:
when the first position is within a threshold distance of the desired trajectory within the multi-dimensional mental state space, causing continued playback of the first media item; and
when the first position is not within the threshold distance of the desired trajectory within the multi-dimensional mental state space: (i) determining a revised trajectory within the multi-dimensional mental state space, wherein the revised trajectory is based on a path from the first position to the target position; (ii) modifying the expected trajectory of the first media item to approximate the revised trajectory by modifying one or more audio parameters of the audio track; and (iii) causing playback of the first media item with the one or more modified audio parameters.

10. The method of claim 9, further comprising:
causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more audio parameters of the audio track.

11. The method of claim 9, wherein modifying one or more audio parameters of the audio track comprises modifying one or more of: (i) a tempo of the audio track, (ii) a RMS (root mean square energy in signal) of the audio track, (iii) a loudness of the audio track, (iv) an event density of the audio track, (v) a spectral brightness of the audio track, (vi) a temporal envelope of the audio track or elements thereof, (vii) a spectral envelope structure of the audio track, (viii) one or more dominant pitches of the audio track, (ix) a self-similarity of the audio track over time, (x) how acoustic energy within the audio track is distributed over spectral modulation rates, (xi) an attack or decay of the audio track that changes a rise and fall time of audio events within the audio track, (xii) textural aspects of the audio track, (xiii) a degree of harmonicity/inharmonicity of the audio track, or (xiv) a sparseness of the audio track.

12. The method of claim 5, wherein the first media item comprises an audio track, wherein the expected trajectory corresponding to the first media item is based on (i) one or more audio parameters of the audio track and (ii) one or more modulation characteristics of the audio track, and wherein the method further comprises:
when the first position is within a threshold distance of the desired trajectory within the multi-dimensional mental state space, causing continued playback of the first media item; and
when the first position is not within the threshold distance of the desired trajectory within the multi-dimensional mental state space: (i) determining a revised trajectory within the multi-dimensional mental state space, wherein the revised trajectory is based on a path from the first position within the multi-dimensional mental state space to the target position within the multi-dimensional mental state space; (ii) modifying the expected trajectory of the first media item to approximate the revised trajectory by modifying one or more modulation characteristics of the audio track; and (iii) causing playback of the first media item with the one or more modified modulation characteristics.

13. The method of claim 12, further comprising:
causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more modulation characteristics of the audio track.

14. The method of claim 12, wherein modifying one or more modulation characteristics of the audio track comprises modifying one or more of: (i) a modulation depth at a single modulation rate; (ii) a modulation rate; (iii) a plurality of modulation depths at a corresponding plurality of modulation rates; (iv) a modulation phase; or (v) a modulation waveform shape.

15. The method of claim 1, wherein the target mental state corresponds to an activity selected from a set of activities comprising: (i) focus, (ii) meditation, (iii) relaxation, and (iv) sleep.

16. Tangible, non-transitory, computer readable media comprising program instructions, wherein the program instructions, when executed by one or more processors, causes a computing system to perform functions comprising:
for a user, determining a desired trajectory within a multi-dimensional mental state space, wherein the desired trajectory is based on a path from an initial position within the multi-dimensional mental state space to a target position within the multi-dimensional mental state space, wherein the initial position corresponds to an initial mental state of the user, and wherein the target position corresponds to a target mental state of the user;
from a media library comprising a plurality of media items, selecting a first media item that has an expected trajectory within the multi-dimensional mental state space that approximates the desired trajectory within the multi-dimensional mental state space, wherein each media item in the media library has a corresponding expected trajectory within the multi-dimensional mental state space; and
causing playback of the first media item.

17. The tangible, non-transitory computer-readable media of claim 16, wherein the functions further comprise:
causing a graphical user interface to display a representation of the multi-dimensional mental state space and one or more of: (i) an indication of the initial position within the multi-dimensional mental state space corresponding to the initial mental state of the user, (ii) an indication of the target position within the multi-dimensional mental state space corresponding to the target mental state of the user; (iii) an indication of the desired trajectory, or (iv) an indication of the expected trajectory corresponding to the first media item.

18. The tangible, non-transitory computer-readable media of claim 16, wherein the functions further comprise, while the first media item is playing:
determining a mental state of the user at a first time, wherein the mental state at the first time corresponds to a first position within the multi-dimensional mental state space;
when the first position is within a threshold distance of the desired trajectory within the multi-dimensional mental state space, causing continued playback of the first media item; and
when the first position is not within the threshold distance of the desired trajectory within the multi-dimensional mental state space, (i) determining a revised trajectory within the multi-dimensional mental state space, wherein the revised trajectory is based on a path from the first position to the target position, at least one of (i-a) selecting a second media item that has an expected trajectory within the multi-dimensional mental state space that approximates the revised trajectory, (i-b) modifying the expected trajectory of the first media item to approximate the revised trajectory by modifying one or more audio parameters of the audio track, or (i-c) modifying the expected trajectory of the first media item to approximate the revised trajectory by modifying one or more modulation characteristics of the audio track, and (iii) causing a transition from playback of the first media item to playback of the second media item.

19. The tangible, non-transitory computer-readable media of claim 18, wherein the functions further comprise:
causing a graphical user interface to display a representation of the multi-dimensional mental state space and one or more of (i) an indication of the desired trajectory, (ii) an indication of the revised trajectory, (iii) an indication of the expected trajectory corresponding to the first media item, or (iv) an indication of a modified trajectory of the first media item after modifying one or more audio parameters of the audio track.

20. The tangible, non-transitory computer-readable media of claim 18, wherein, wherein:
modifying one or more audio parameters of the audio track comprises modifying one or more of: (i) a tempo of the audio track, (ii) a RMS (root mean square energy in signal) of the audio track, (iii) a loudness of the audio track, (iv) an event density of the audio track, (v) a spectral brightness of the audio track, (vi) a temporal envelope of the audio track or elements thereof, (vii) a spectral envelope structure of the audio track, (viii) one or more dominant pitches of the audio track, (ix) a self-similarity of the audio track over time, (x) how acoustic energy within the audio track is distributed over spectral modulation rates, (xi) an attack or decay of the audio track that changes a rise and fall time of audio events within the audio track, (xii) textural aspects of the audio track, (xiii) a degree of harmonicity/inharmonicity of the audio track, or (xiv) a sparseness of the audio track; and
modifying one or more modulation characteristics of the audio track comprises modifying one or more of: (i) a modulation depth at a single modulation rate; (ii) a modulation rate; (iii) a plurality of modulation depths at a corresponding plurality of modulation rates; (iv) a modulation phase; or (v) a modulation waveform shape.
